(19) United States
(12) Patent Application Publication
(10) Pub. No.: US 2025/0050072 A1
(43) Pub. Date: Feb. 13, 2025

(54) DEVICE, SYSTEM, AND METHOD OF VENOUS ACCESS

(71) Applicant: The Johns Hopkins University, Baltimore, MD (US)

(72) Inventors: Hussam Eldin Mohamed Adam Mohamed Shwaib, Baltimore, MD (US); Nicole Chandler, Baltimore, MD (US)

(21) Appl. No.: 18/719,932

(22) PCT Filed: Dec. 14, 2022
(86) PCT No.: PCT/US2022/052833

§ 371 Date: Jun. 14, 2024

Related U.S. Application Data
(60) Provisional application No. 63/289,700, filed on Dec. 15, 2021.

(51) Int. Cl. A61M 25/09; A61M 25/06; A61M 29/00

(52) U.S. Cl. CPC A61M 25/09; A61M 25/065; A61M 29/00; A61M 2205/52

(57) ABSTRACT

A catheter deployment device including a housing; and a panel coupled to the housing. The panel extends from the housing at an angle and the panel includes an aperture. The deployment device further includes a deployment assembly coupled to the housing. The deployment assembly is aligned with the aperture along an insertion axis.

### CROSS REFERENCE TO RELATED APPLICATIONS

This application claims priority to and the benefit of U.S. Provisional Patent Application No. 63/289,700 filed on Dec. 15, 2021 and is incorporated herein by reference in its entirety for all purposes.

### BACKGROUND

Central venous access is a common medical procedure where catheters are placed in a vein of a patient. Central venous access is a form of venous access, more generally, that focuses on the placement of catheters in centrally located veins and is typical used as a more reliable vascular access for prolonged intravenous therapies or critically ill patients.

Conventional central venous access procedures follow the Seldinger technique, which is generally illustrated in FIG. 1. See An Introduction to Clinical Emergency Medicine, Cambridge University Press, 2012. However, conventional procedures have a considerable rate of variation in terms of complication rates. For example, one review described an overall complication rate with conventional central venous access procedures of 15 percent. The rate of mechanical or procedure-related complications, is mainly operator dependent. Published rates of cannulation success and complications vary according to the anatomic site, the use of ultrasound guidance, and operator experience. Mechanical complications can occur in up to 33 percent of cannulation attempts with inexperienced operators. Examples of mechanical complications include bleeding, arterial puncture, arrhythmia, air embolism, thoracic duct injury, nerve injury, catheter malposition, and pneumothorax or hemothorax.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure provides in one aspect, a deployment device including a housing and a panel coupled to the housing. The panel extends from the housing at an angle and the panel includes an aperture. The deployment device further includes a deployment assembly coupled to the housing. The deployment assembly is aligned with the aperture along an insertion axis.

In some embodiments, the deployment device further includes an actuator configured to move a portion of the deployment assembly relative to the housing.

In some embodiments, the actuator is configured to move the portion along the insertion axis.

In some embodiments, the actuator is configured to move the portion along a transverse axis that intersects the insertion axis.

In some embodiments, the transverse axis is perpendicular to the insertion axis.

In some embodiments, the actuator is a first actuator and the portion is a first portion, and wherein the device further includes a second actuator configured to move a second portion of the deployment assembly along a transverse axis that is perpendicular to the insertion axis.

In some embodiments, the actuator is a first actuator and the portion is a first portion, and wherein the device further includes a second actuator configured to move a second portion of the deployment assembly relative to the housing.

In some embodiments, the actuator is an electric motor and the device further includes a power supply positioned within the housing.

In some embodiments, the housing includes a surface with an opening, and wherein the panel extends from the surface and the opening is aligned with the aperture.

In some embodiments, the angle is within a range of 30 degree to 90 degrees.

In some embodiments, the angle is 90 degrees.

In some embodiments, the deployment assembly is a peripheral venous access deployment module.

In some embodiments, the deployment assembly is a central venous access deployment module.

In some embodiments, the central venous access deployment module includes a needle, a dilator, a guidewire, and a catheter.

In some embodiments, the central venous access deployment module includes a retractable knife.

In some embodiments, the deployment device further includes a processor and a memory, wherein the memory includes instructions that when executed by the processor position the catheter within a vein of a patient.

In some embodiments, the deployment assembly is a first deployment assembly and the first deployment assembly is removable from the housing and replaceable with a second deployment assembly.

In some embodiments, the actuator is coupled to the deployment assembly by a detachable interface.

In some embodiments, the deployment device further includes an ultrasound module coupled to the panel.

In some embodiments, the deployment device further includes a handle coupled to the housing.

The present disclosure provides, in one aspect, an assembly including a base with a frame and a carrier movable with respect to the frame. The assembly further includes a guidewire extending along an insertion axis and movable with respect to the frame along the insertion axis. The assembly further includes a needle module coupled to the carrier, and a catheter movable with respect to the frame along the insertion axis.

In some embodiments, the needle module is movable with the carrier, and movable with respect to the carrier along the insertion axis.

In some embodiments, the needle module includes a slide, a needle coupled to the slide, an actuator, and a transmission positioned between the actuator and the slide, and wherein the actuator is activated to move the needle along the insertion axis.

In some embodiments, the actuator is activated to move the needle relative to the carrier along the insertion axis.

In some embodiments, the needle module further includes a cover movable between a closed configuration and an open configuration.

In some embodiments, the assembly further includes a cover actuator coupled to the cover, wherein the cover actuator is activated to move the cover between the closed configuration and the open configuration.

In some embodiments, the needle includes a slot. The cover is spaced from the slot when the needle is in the open configuration.

In some embodiments, the carriage moves relative to the frame in a direction transverse to the insertion axis.

In some embodiments, the needle module includes a knife movable along the insertion axis.

In some embodiments, the knife is movable with respect to the guidewire.

In some embodiments, the needle module further includes a dilator.

In some embodiments, the dilator is movable with respect to the guidewire along the insertion axis.

In some embodiments, the needle module includes a needle, a dilator, and a knife.

In some embodiments, the needle is independently movable with respect to the dilator and the knife, and the knife is independently movable with respect to the needle and the dilator.

In some embodiments, the needle is at least partially positioned within the dilator, and the knife is at least partially positioned within the dilator.

In some embodiments, the dilator includes a dilator slot aligned with a slot formed in the needle.

In some embodiments, the assembly further includes a guidewire drive, wherein actuation of the guidewire drive moves the guidewire along the insertion axis.

In some embodiments, the assembly further includes a catheter drive, wherein actuation of the catheter drive moves the catheter along the insertion axis.

In some embodiments, the assembly further includes a second carrier movable with respect to the frame along the insertion axis. The guidewire drive and the catheter drive are coupled to the second carrier.

In some embodiments, the guidewire is positioned within the catheter.

The present disclosure provides in one aspect, a device including a cylindrical member with a cylindrical wall extending along a longitudinal axis. A slot is formed in the cylindrical wall and the slot extends along the longitudinal axis. The device further includes a blocking member movable with respect to the cylindrical member between a closed configuration in which the slot is blocked by the blocking member and an open configuration in which the slot is open to the longitudinal axis.

In some embodiments, the cylindrical member is a first cylindrical member and the cylindrical wall is a first cylindrical wall. The blocking member is a second cylindrical member with a second cylindrical wall extending along the longitudinal axis.

In some embodiments, the second cylindrical wall is positioned within the first cylindrical wall.

In some embodiments, the slot is a first slot and the second cylindrical member includes a second slot. The first slot and the second slot are aligned in the open configuration, and the first slot and the second slot are misaligned in the closed configuration.

In some embodiments, the first cylindrical wall includes a first outer surface and a first inner surface, and the first slot extends between the first outer surface and the first inner surface. The second cylindrical wall includes a second outer surface and a second inner surface, and the second slot extends between the second outer surface and the second inner surface.

In some embodiments, the first inner surface is positioned between the first outer surface and the second outer surface.

In some embodiments, the second cylindrical member includes an actuator radially extending from the second cylindrical wall.

In some embodiments, the actuator extends through an aperture formed in the first cylindrical wall.

In some embodiments, the device further includes a handle coupled to an end of the first cylindrical member, and the actuator extends through the handle.

In some embodiments, the handle includes a handle slot aligned with the first slot.

In some embodiments, the device further includes a biasing member positioned between the first cylindrical member and the second cylindrical member. The biasing member biases the second cylindrical member toward the closed configuration.

In some embodiments, a first end of the biasing member abuts a stop formed on the first cylindrical member, and a second end of the biasing member abuts a radial wall of the second cylindrical member.

In some embodiments, the device further includes a biasing member positioned between the cylindrical member and the blocking member. The biasing member biases the blocking member toward the closed configuration.

In some embodiments, the first cylindrical member includes a support hub coupled to the second cylindrical member.

In some embodiments, the support hub includes a radial portion extending radially inward from the first cylindrical wall and a bearing portion.

In some embodiments, the radial portion extends through the second cylindrical wall.

In some embodiments, the device is a needle.

In some embodiments, the cylindrical member includes a beveled distal end.

In some embodiments, the device is a dilator.

In some embodiments, the cylindrical member includes a conical distal end.

In some embodiments, the device in the open configuration is configured to radially receive a guidewire through the slot.

Other aspects of the disclosure will become apparent by consideration of the detailed description and accompanying drawings.

### BRIEF DESCRIPTION OF THE DRAWINGS

The accompanying figures and examples are provided by way of illustration and not by way of limitation. The foregoing aspects and other features of the disclosure are explained in the following description, taken in connection with the accompanying example figures (also “FIG.”) relating to one or more embodiments.

The patent or application file contains at least one drawing executed in color. Copies of this patent or patent application publication with color drawings will be provided by the Office upon request and payment of the necessary fee.

FIG. 1 is a schematic illustrating a conventional central venous access procedure.

FIG. 2 is a perspective view of a deployment device with a deployment assembly.

FIG. 3 is another perspective view of the deployment device of FIG. 2.

FIG. 4 is a cross-sectional view of the deployment device of FIG. 3.

FIG. 5 is a perspective view of a deployment device and a deployment assembly, with portions removed for clarity.

FIG. 6 is a perspective cross-sectional view of the deployment device and deployment assembly of FIG. 2.

FIG. 7A is a perspective view of a deployment device and deployment assembly, illustrated in a starting configuration.

FIG. 7B is a perspective view of a deployment device and deployment assembly, illustrating a needle module in an inserted position.

FIG. 7C is a perspective view of the deployment device and deployment assembly, illustrating the needle module returned from the inserted position.

FIG. 7D is a perspective view of the deployment device and deployment assembly, illustrating the needle module in a separated configuration.

FIG. 7E is a perspective view of the deployment device and deployment assembly, illustrating a dilator module in an inserted position.

FIG. 7F is a perspective view of the deployment device and deployment assembly, illustrating the dilator module returned from the inserted position.

FIG. 7G is a perspective view of the deployment device and deployment assembly, illustrating the dilator module in a separated configuration.

FIG. 8 is a partial perspective view of the deployment device and deployment assembly, illustrating a catheter and catheter drive wheel.

FIG. 9 is a perspective view of a base module of the deployment assembly.

FIG. 10 is FIG. 9 with portions shown transparently for clarity.

FIG. 11 is a perspective view of a needle module in a first configuration.

FIG. 12 is another perspective view of the needle module of FIG. 11.

FIG. 13 is a cross-sectional view of the needle module of FIG. 11.

FIG. 14 is a perspective view of the needle module of FIG. 11 in a second, separated configuration.

FIG. 15 is a perspective view of a dilator module in a first configuration.

FIG. 16 is a cross-sectional view of the dilator module of FIG. 15.

FIG. 17 is a perspective view of the dilator module of FIG. 15 in a second, separated configuration.

FIG. 18 is a perspective view of a needle including a cylindrical member and a blocking member.

FIG. 19 is a perspective view of a dilator including a cylindrical member and a blocking member.

FIG. 20A is a perspective cross-sectional view of the needle of FIG. 18 with portions removed for clarity and a guidewire positioned within the needle, wherein the needle is illustrated in a closed configuration.

FIG. 20B is a perspective cross-sectional view of the needle and guidewire of FIG. 20A, wherein the needle is illustrated in an open configuration.

FIG. 20C is a perspective cross-sectional view of the needle and guidewire of FIG. 20A, wherein the needle is illustrated in an open configuration and the guidewire is removed from the needle.

FIG. 21 is a schematic comparing operation of the needle of FIG. 18 and a conventional needle.

FIG. 22 is a schematic comparing operation of the dilator of FIG. 19 and a conventional dilator.

FIG. 23 is a perspective view of a deployment device with a deployment assembly.

FIG. 24 is a perspective view of the deployment assembly of FIG. 23.

FIG. 25 is a cross-sectional view of the deployment device and the deployment assembly of FIG. 23.

FIG. 26 is a perspective view of a needle module including a needle, a dilator, a knife, and a movable cover.

FIG. 27 is an exploded view of the needle module of FIG. 26.

FIG. 28 is a cross-sectional view of the needle module of FIG. 26.

FIG. 29 is a perspective view of a cross-section of a catheter module.

FIG. 30 is a perspective view of a base with a frame, a first carrier for the needle module of FIG. 26, and a second carrier for the catheter module.

FIG. 31 is a perspective view of a guidewire drive and a catheter drive.

FIGS. 32A-G illustrate operation of the deployment device of FIG. 23.

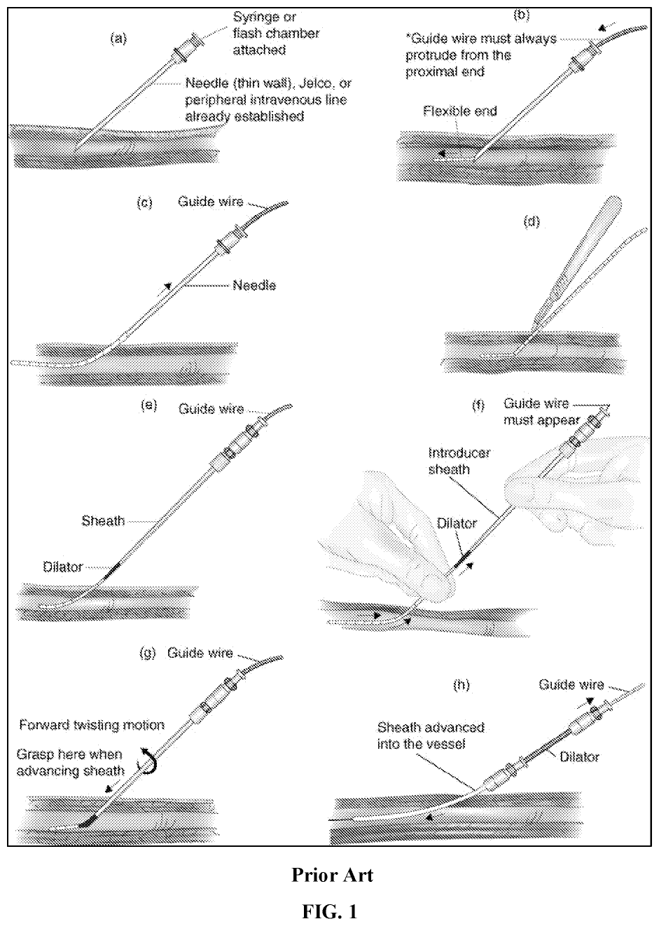

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” and “approximately” are used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations when interpreted in the alternative (“or”).

Moreover, the present disclosure also contemplates that in some embodiments, any feature or combination of features set forth herein can be excluded or omitted. To illustrate, if the specification states that an apparatus comprises components A, B, and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

According to one aspect of the present disclosure, a universal deployment device utilizes single-use replaceable deployment assemblies to provide a handheld, battery-powered device that automates central venous access (CVA).

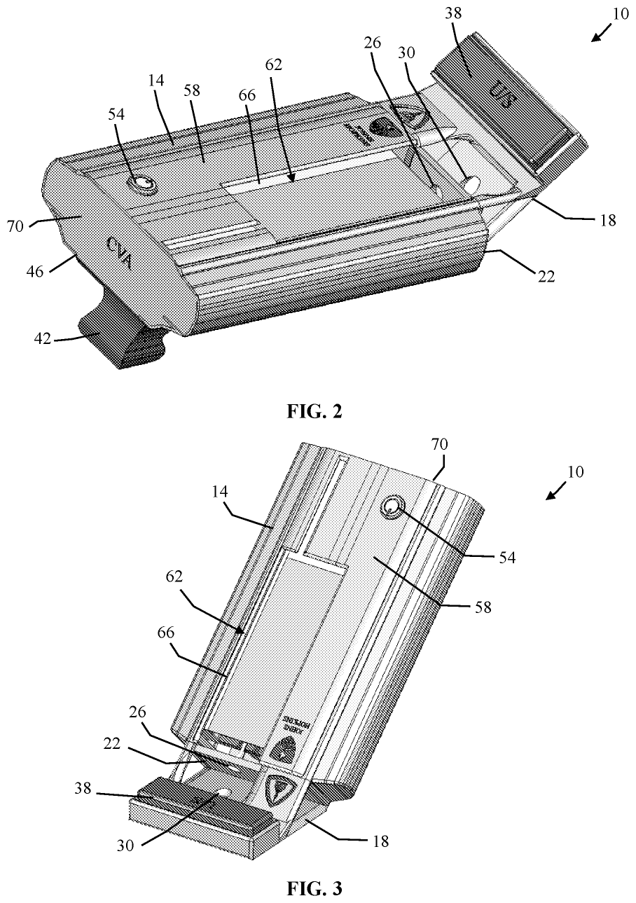

With reference to FIGS. 2 and 3, a deployment device 10 (i.e., a universal deployment device) is illustrated with a housing 14 and a panel 18 coupled to the housing 14. The housing 14 includes a surface 22 with a first aperture 26 formed therein. The panel 18 includes a second aperture 30 and the panel 18 extends from the surface 22 of the housing 14 at an angle 34. In some embodiments, the angle 34 is within a range of approximately 30 degrees to approximately 50 degrees. In some embodiments, the angle 34 is approximately 40 degrees. In some embodiments, an ultrasound module 38 is coupled to the panel 18 and is utilized to aid positioning of the deployment device 10 relative to a patient.

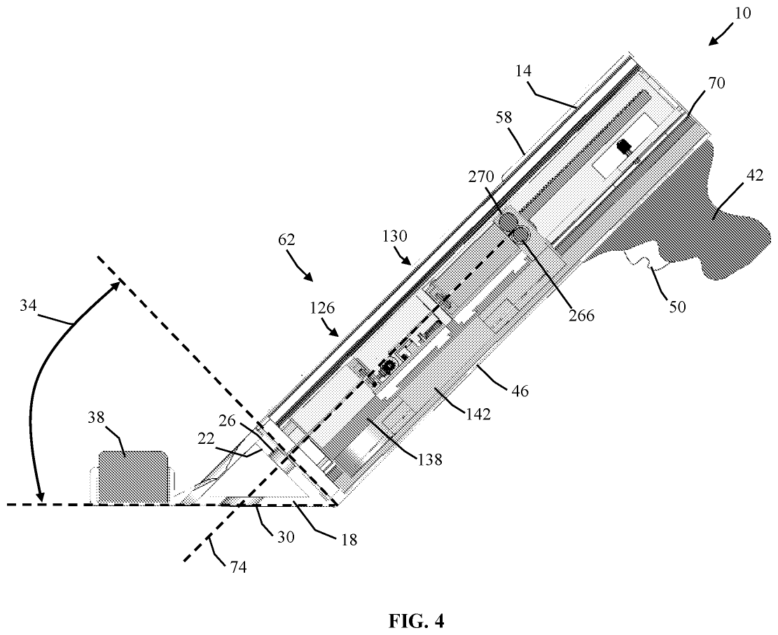

In some embodiments, the deployment device 10 includes a handle 42 coupled to the housing 14. In the illustrated embodiment, the handle 14 extends from a lower surface 46 of the housing 14. As such, the deployment device 10 can be considered a hand-held device. In some embodiments, user-activated controls 50 (FIG. 4) are positioned on the handle 42. In some embodiments, user-activated controls 54 (FIG. 2) are positioned on a top surface 58 of the housing 14. In the illustrated embodiment, the top surface 58 is positioned opposite from the lower surface 46.

With continued reference to FIGS. 2 and 3, the deployment device 10 further includes a deployment assembly 62 coupled to the housing 14. In the illustrated embodiment, the deployment assembly 62 is received within a cutout 66 formed in the housing 14. In the illustrated embodiment, the cutout 66 is positioned closer to the front surface 22 than to a rear surface 70 of the housing 14. The deployment assembly 62 is aligned with the first aperture 26 and the second aperture 30 along an insertion axis 74 (FIG. 6). In other words, the insertion axis 74 passes through the first aperture 26, the second aperture 30, and the deployment assembly 62 (FIG. 4). As described in greater detail herein, the deployment device 10 includes an actuator 81 configured to move a portion of the deployment assembly 62 relative to the housing 14.

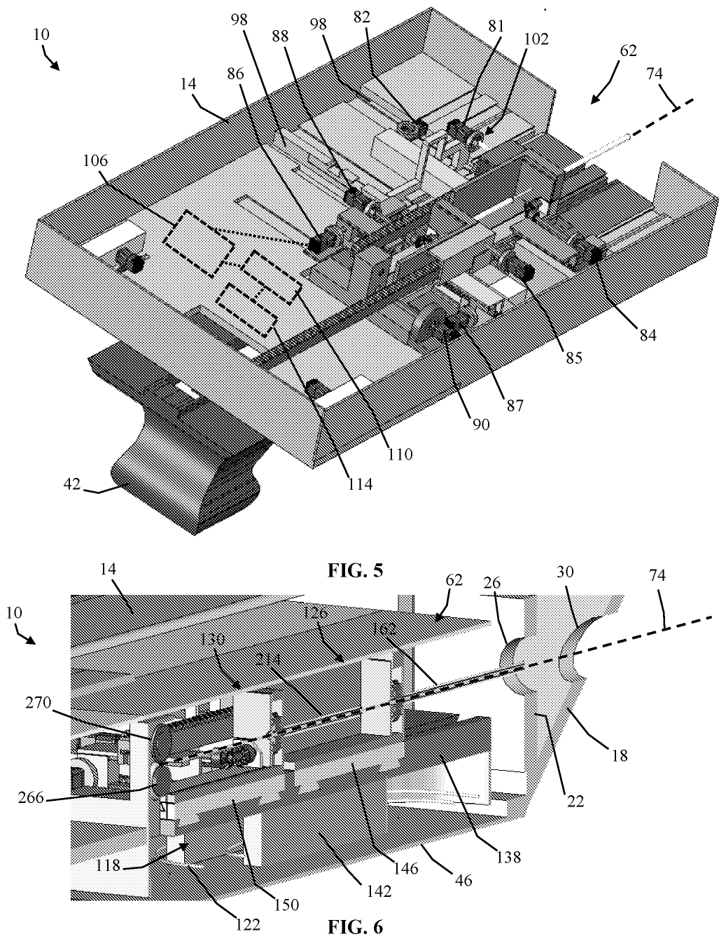

With reference to FIG. 5, the deployment device 10 includes a plurality of actuators 81, 82, 83, 84, 85, 86, 87, 88, 89, and 90. In some embodiments, the plurality of actuators 81-90 are configured to move various portions of the deployment assembly 62. In the illustrated embodiment, the deployment device 10 includes ten actuators 81-90. In some embodiments, the plurality of actuators 81-90 is a plurality of electric motors. As explained in greater detail herein, the plurality of actuators 81-90 is configured to move various portions of the deployment assembly 62 relative to the housing 14. In the illustrated embodiment, the actuator 81 is configured move a portion of the deployment assembly 10 along the insertion axis 74, whereas the actuators 82 and 83 are configured to move portions of the deployment assembly 72 along a transverse axis (e.g., carrier axis 198, carrier axis 250) that intersects the insertion axis 74. In some embodiments, the transverse axis is perpendicular to the insertion axis 74. The housing 14 includes at least one slot 92 formed therein. In the illustrated embodiments, a portion of the deployment assembly 62 is movable relative to the housing 14 within the slot 98.

The deployment assembly 62 is removable from the housing 14 and is replaceable with another, different deployment assembly. In the illustrated embodiment, the deployment assembly 62 is removably coupled to the cutout 66 in the housing 14. In some embodiments, the deployment assembly 62 is a single-use assembly intended for use in a single procedure. As explained in greater detail herein, a detachable interface 102 couples the actuators 81-90 to the removable deployment assembly 62. For example, the detachable interface 102 includes a groove 106 formed in the actuator 81 and a drive shaft 106 in the deployment assembly 62 at least partially positioned within the groove 106. In the illustrated embodiment, the detachable interface 102 is a key in slot removable rotational coupling.

In some embodiments, the deployment device 10 is configured to receive different types of deployment assemblies. In some embodiments, the deployment assembly 62 is a peripheral venous access deployment module. In some embodiments, the deployment assembly 62 is a central venous access deployment module. In some embodiments, the deployment assembly 62 is a spinal or epidural anesthesia deployment module. In other words, the same deployment device 10 can be utilized for deployment assemblies tailored to a variety of medical procedures. As such, in some embodiments, the deployment assembly 62 is a first deployment assembly and the first deployment assembly is removable from the housing 14 by a user and replaceable with a second deployment assembly. In some embodiments, the first deployment assembly is used with a first medical procedure and the second deployment assembly is used with a second medical procedure.

With reference to FIG. 5, the deployment device 10 includes a power supply 106, a processor 110, and a memory 114 positioned within the housing 14. In some embodiments, the power supply 106 is a removable and rechargeable battery pack. In other embodiments, the power supply 106 includes a corded AC power cord. In the illustrated embodiment, the power supply 106 is electrically coupled to the processor 110 and at least one of the actuators 81-90, and the memory 114 is electrically coupled to the processor 110.

The processor 110 (e.g., a microprocessor, a microcontroller, a processing unit, or other suitable programmable device) can include, among other things, a control unit, an arithmetic logic unit (“ALC”), and a plurality of registers, and can be implemented using a known computer architecture (e.g., a modified Harvard architecture, a von Neumann architecture, etc.). In some embodiments the processor 110 is a microprocessor that can be configured to communicate in a stand-alone and/or a distributed environment, and can be configured to communicate via wired or wireless communications with other processors, where such one or more processor can be configured to operate on one or more processor-controlled devices that can be similar or different devices.

In some embodiments, the memory 114 is any memory storage and is a non-transitory computer readable medium. The memory can include, for example, a program storage area and the data storage area. The program storage area and the data storage area can include combinations of different types of memory, such as a ROM, a RAM (e.g., DRAM, SDRAM, etc.), EEPROM, flash memory, a hard disk, a SD card, or other suitable magnetic, optical, physical, or electronic memory devices.

The processor 110 can be connected to the memory 114 and execute software instructions that are capable of being stored in a RAM of the memory (e.g., during execution), a ROM of the memory (e.g., on a generally permanent bases), or another non-transitory computer readable medium such as another memory or a disc. In some embodiments, the memory 114 includes one or more processor-readable and accessible memory elements and/or components that can be internal to the processor-controlled device, external to the processor-controlled device, and can be accessed via a wired or wireless network. Software included in the implementation of the methods disclosed herein can be stored in the memory. The software includes, for example, firmware, one or more applications, program data, filters, rules, one or more program modules, and other executable instructions. For example, the processor 110 can be configured to retrieve from the memory and execute, among other things, instructions related to the processes and methods described herein. For example, in some embodiments, the memory 114 includes instructions that when executed by the processor 110, activate at least one of the actuators 81-90 to automatically provide central venous access in a patient (e.g., to position a catheter within a vein of a patient).

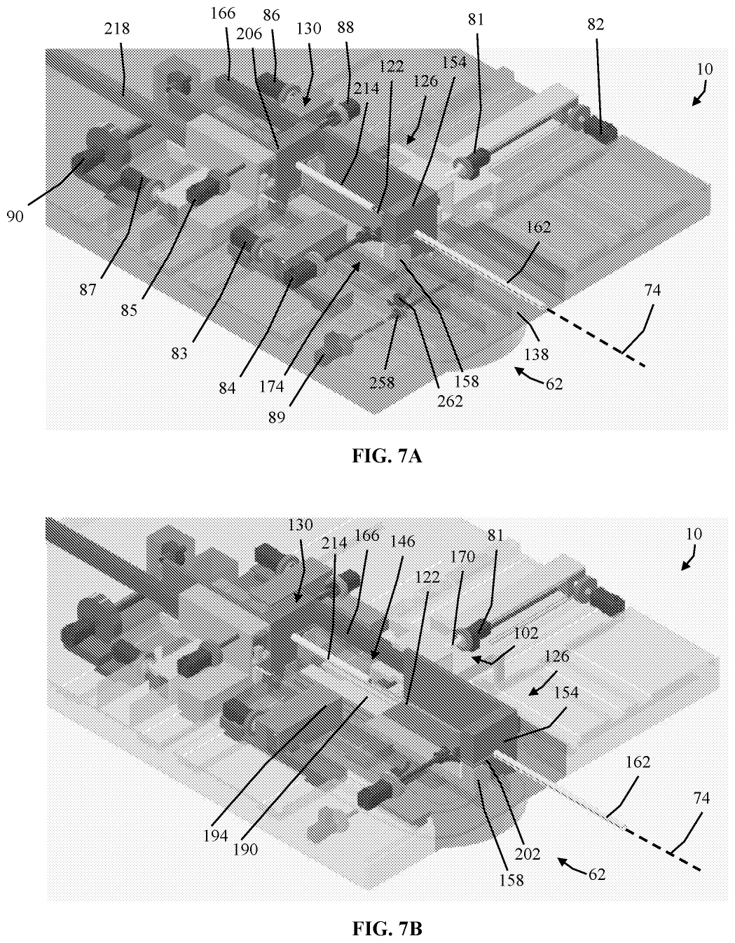

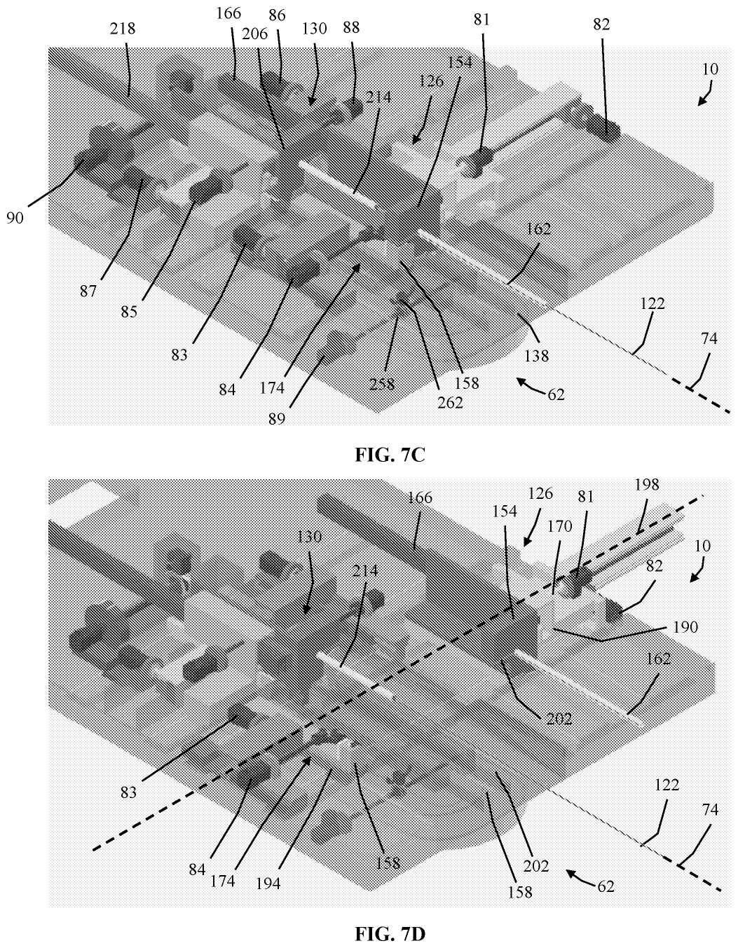

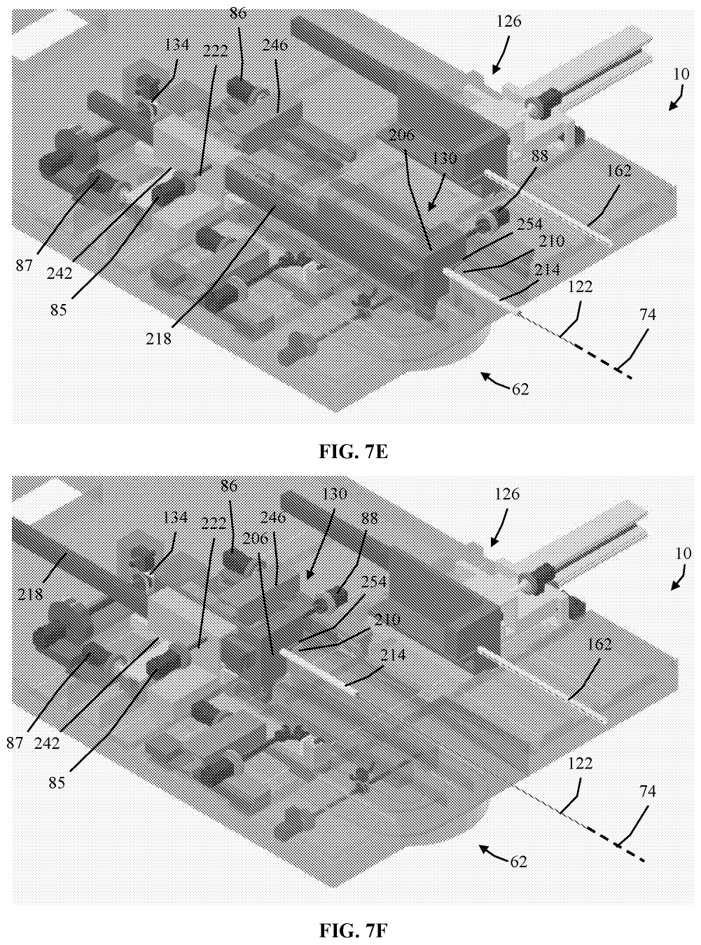

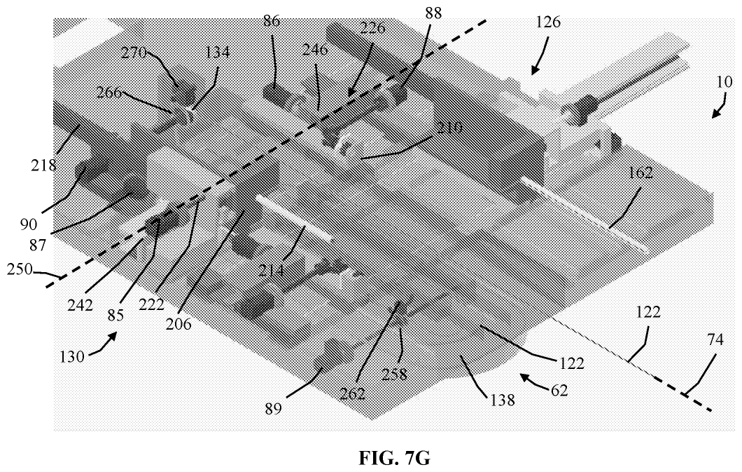

The description now turns to the details of the deployment assembly 62, illustrated as a central venous access deployment assembly. With reference to FIGS. 7A-7G, the deployment assembly 62 includes a base module 118, a guidewire 122, a needle module 126, a dilator module 130, and a catheter 134. The guidewire 122 extends along the insertion axis 74 and is movable with respect to the base module 118 along the insertion axis 74. Likewise, the catheter 134 is movable with respect to the base module 118 along the insertion axis 74. In particular, the guidewire 122 and the catheter 134 move with respect to a base plate 138 of the base module 118 along the insertion axis 74. In some embodiments, a portion of the guidewire 122 is positioned underneath the base plate 138. In particular, a portion of the guidewire 122 is positioned within a storage chamber 142 formed in the base plate 138.

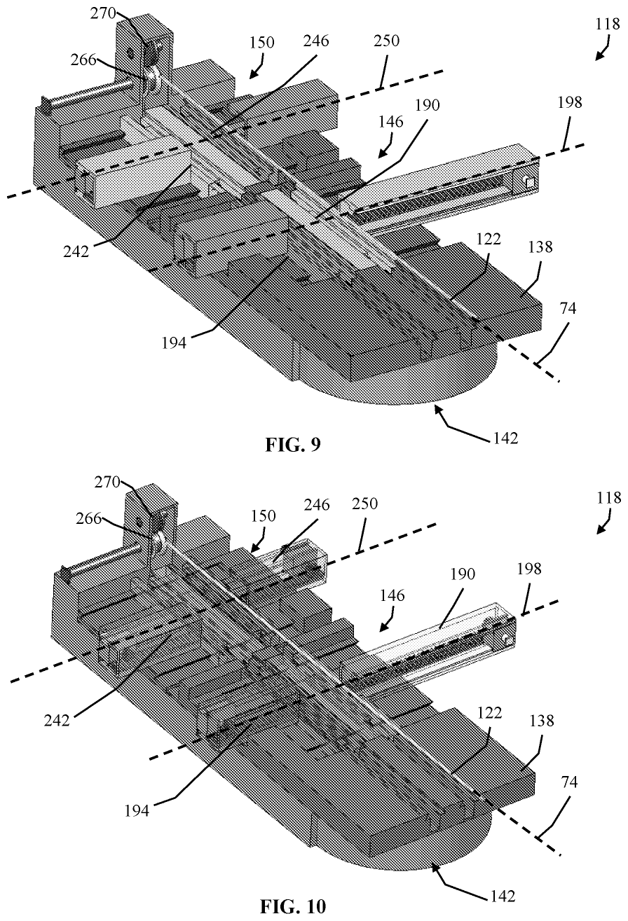

With reference to FIGS. 9 and 10, the base module 118 includes the base plate 138, a first carrier 146 (e.g., a needle carrier) movable with respect to the base plate 138, and a second carrier 150 (e.g., a dilator carrier) movable with respect to the base plate 138. In the illustrated embodiment, the first carrier 146 is coupled to the needle module 126 and the second carrier 150 is coupled to the dilator module 130. In some embodiments, the needle module 126 is movable with the first carrier 146 and the dilator module 130 is movable with the second carrier 150.

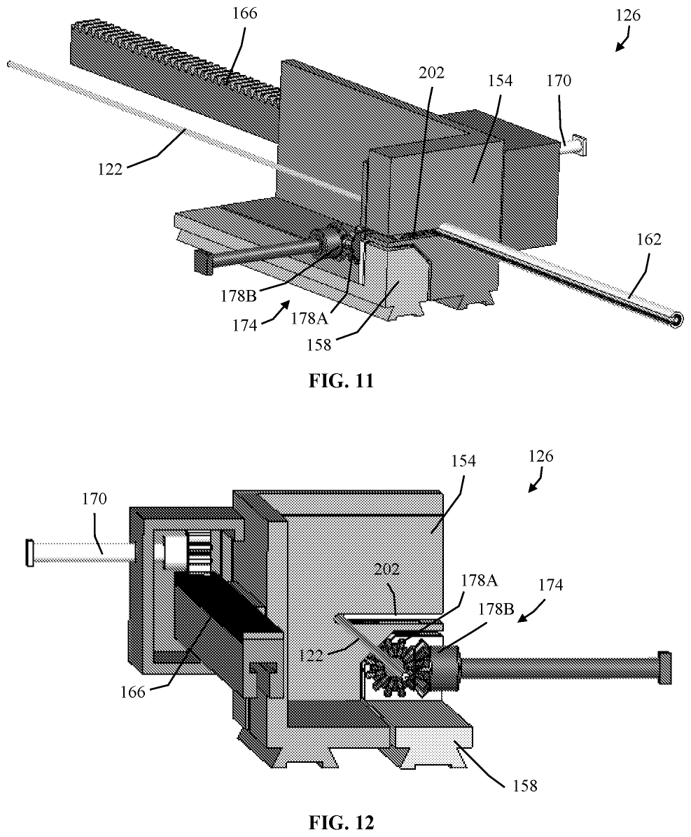

With reference to FIGS. 11-14, the needle module 126 includes a first slide 154, a second slide 158 separable from the first slide 154, a needle 162 coupled to the first slide 154, and a rack 166 coupled to the first slide 154. A transmission 170 is positioned between the actuator 81 and the rack 166. In the illustrated embodiment, the transmission 170 is removably coupled to the actuator 81 by the detachable interface 102. In the illustrated embodiment, the actuator 81 is activated to move the needle 162 (and the slides 154, 158) along the insertion axis 74. In some embodiments, the needle module 126 is movable with respect to the needle carrier 146 along the insertion axis 74. During operation, the needle module 126 is driven forward along the insertion axis 74 (i.e., from FIG. 7A to FIG. 7B) and then the needle module 126 is driven backwards along the insertion axis 74 (i.e., from FIG. 7B to FIG. 7C).

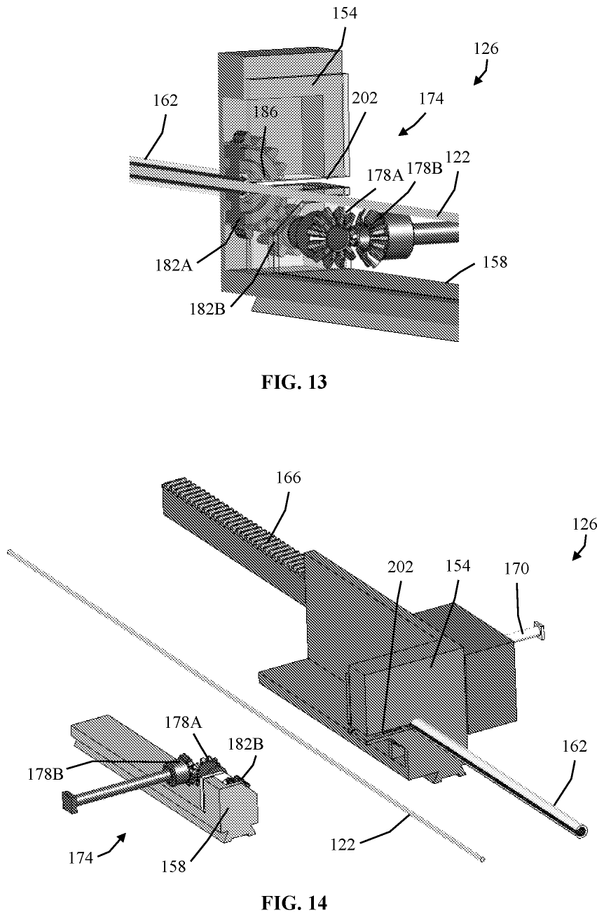

With continued reference to FIGS. 11-14, the needle 162 is movable between a closed configuration (FIG. 11) and an open configuration (FIG. 14). A transmission 174 is positioned between the actuator 84 and the needle 162. In the illustrated embodiment, the transmission 174 is coupled to the second slide 158. The actuator 84 is activated to move the needle 162 between the closed configuration in which the needle 162 is not laterally separable from the guidewire 122 and the open configuration in which the needle 162 is laterally separable from the guidewire 122. With reference to FIG. 13, the transmission 174 in the illustrated embodiment includes a bevel gear set 178A, 178B and a spur gear set 182A, 182B. The spur gear set 182A, 182B includes a first gear 182A positioned on the first slide 154 and a second gear 182B positioned on the second slide 158. In the illustrated embodiment, at least a portion of the needle 162 is rotatably coupled to the first gear 182A. With reference to FIG. 13, the first gear 182A includes a slot 186 through which the guidewire 122 is permitted to move.

As explained in greater detail herein, the needle 162 is initially positioned around the guidewire 122 in the closed configuration (through FIGS. 7A-7C). When the needle 162 is moved to the open configuration (FIG. 7D) and the needle 162 is able to move laterally with respect to the guidewire 122 and the insertion axis 74. In this way, the needle 162 is advantageously quickly removed from the insertion axis 162 after use to permit other modules (e.g., the dilator module 130) to move along the insertion axis 74.

With reference to FIG. 9, the first carrier 146 includes a first mount 190 (i.e., a first carrier portion) coupled to the first slide 154 and a second mount 194 (i.e., a second carrier portion) coupled to the second slide 158. The first mount 190 and the second mount 194 are movable along a carrier axis 198 (i.e., a transverse axis) between a first configuration (FIG. 7C) with the first slide 154 and the second slide 158 of the needle module 126 coupled together and a second configuration (FIG. 7D) with the first slide 154 separated from the second slide 158. In other words, in the first configuration (FIG. 7C) the mounts 190, 194 are positioned together and in the second configuration (FIG. 7D) the mounts 190, 194 are spaced apart. In the illustrated embodiment, the first gear 182A and the second gear 182B are enmeshed when the mounts 190, 194 are in the first configuration. The needle 162 is aligned with the insertion axis 74 when the first mount 190 and the second mount 194 are in the first configuration (FIG. 7C) and the needle 162 is separated from the insertion axis 74 when the first mount 190 and the second mount 194 are in the second configuration (FIG. 7D). In some embodiments, the insertion axis 74 is positioned between the mounts 190, 194 when in the second configuration. In some embodiments, the carrier axis 198 is transverse to the insertion axis 74. In some embodiments, the carrier axis 198 is perpendicular to the insertion axis 74. In the illustrated embodiment, the first slide 154 is movable with respect to the first mount 190 along the insertion axis 74 (see FIG. 7B) and the first slide 154 is movable with the first mount 190 along the carrier axis 198 (see FIG. 7D).

With continued reference to FIGS. 11-14, the first slide 154 includes a groove 202 through which the guidewire 122 is allowed to pass as the first slide 154 moves along the carrier axis 198 to the second configuration. The guidewire 122 is positioned within the groove 202 (FIGS. 11 and 12) when the first mount 190 and the second mount 194 are in the first configuration. The guidewire 122 is spaced from the groove 202 (FIG. 14) when the first mount 190 and the second mount 194 are in the second configuration and the slides 154, 158 are spaced apart.

Advantageously, moving the needle laterally with respect to the insertion axis allows the needle to be removed more quickly. After a needle is used in conventional processes, the needle is backed up the entire length of the guidewire to remove the needle from the guidewire. When the guidewire length is long, it is difficult for a single operator to remove the needle from the entire length of guidewire. Some conventional processes therefore need more than one operator to remove a needle from a long guidewire.

Guidewires are used to guide other vascular tools into vessels during vascular procedures, for example. The guidewires vary in dimensions and can have a diameter within a range of approximately 0.014 inches to approximately 0.038 inches and a length within a range of approximately 20 inches to approximately 102 inches. The conventional Seldinger technique for central venous access relies upon the sequential exchange of several vascular tools (e.g., a needle, a dilator, etc.) over the guidewire. The sequential exchange of tools creates challenges and disadvantages for the conventional process. The first disadvantage of the conventional manual central venous access procedure is with threading the vascular tools onto the guidewire. Several attempts may be needed before the operator is successful in manually threading the tool onto the guidewire. The second disadvantage of the conventional manual central venous access procedure is that any exchanged tool needs to pass along the entire length of the guidewire, starting from the distal end and moving toward the patient. Because the guidewires can be long, exchanging tools on the guidewire can require assistance from another operator during the procedure. For example, the surgeon will stand near the proximal end of the guidewire while an assistant stands at the distal end, and the assistant inserts the tool from the distal end and slides the tool over the guidewire until the surgeon receives it at the proximal end for the intended use. After use of the tool is complete, the surgeon then returns the tool to the assistant in the reverse direction. Operation time for the conventional process is longer because to use a vascular tool it must be threaded onto the guidewire and passed along the entire length of the guidewire. Likewise, to remove a vascular tool from the guidewire, it must pass along the entire length of the guidewire again, in reverse. As such, the process of exchanging the tools from the guidewire in the conventional procedure increases the operating time, creates potential hazards for injury, tool slippage, kinking, and other complications.

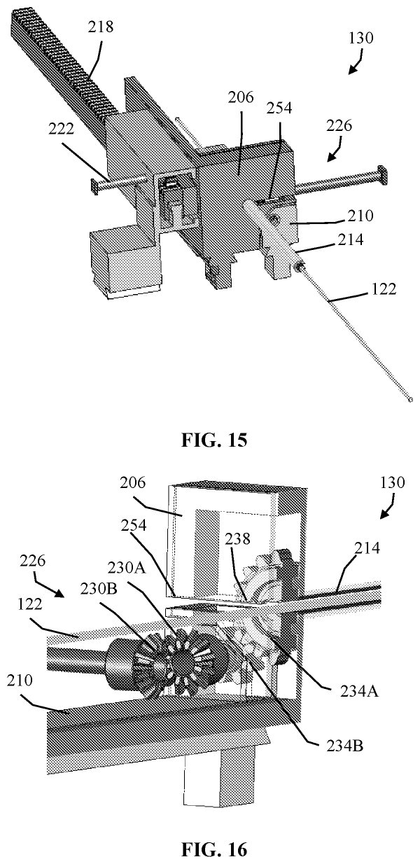

With reference to FIGS. 15-17, the dilator module 130 includes a first slide 206, a second slide 210, a dilator 214 coupled to the first slide 206, and a rack 218 coupled to the first slide 206. A transmission 222 is positioned between the actuator 85 and the rack 218. In the illustrated embodiment, the transmission 222 is removably coupled to the actuator 85 by the detachable interface 102. In the illustrated embodiment, the actuator 85 is activated to move the dilator 214 (and the slides 206, 210) along the insertion axis 74. In some embodiments, the dilator module 130 is movable with respect to the dilator carrier 150 along the insertion axis 74. During operation, the dilator module 130 is driven forward along the insertion axis 74 (i.e., from FIG. 7D to FIG. 7E) and then the dilator module 130 is driven backwards along the insertion axis 74 (i.e., from FIG. 7E to FIG. 7F).

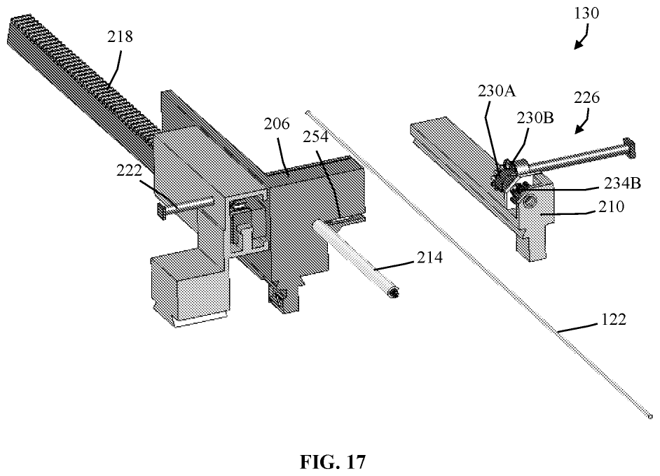

With continued reference to FIGS. 15-17, the dilator 214 is movable between a closed configuration (FIG. 15) and an open configuration (FIG. 17). A transmission 226 is positioned between the actuator 88 and the dilator 214. In the illustrated embodiment, the transmission 226 is coupled to the second slide 210. The actuator 88 is activated to move the dilator 214 between the closed configuration in which the dilator 214 is not laterally separable from the guidewire 122 and the open configuration in which the dilator 214 is laterally separable from the guidewire 122. With reference to FIG. 16, the transmission 226 in the illustrated embodiment includes a bevel gear set 230A, 230B and a spur gear set 234A, 234B. The spur gear set 234A, 234B includes a first gear 234A positioned on the first slide 206 and a second gear 234B positioned on the second slide 210. In the illustrated embodiment, at least a portion of the dilator 214 is rotatably coupled to the first gear 234A. With reference to FIG. 13, the first gear 234A includes a slot 238 through which the guidewire 122 is permitted to move.

In the illustrated embodiment, the first carrier 146 is a needle carrier, and the second carrier 150 is a dilator carrier. Similar to the first carrier 146, the second carrier 150 is movable with respect to the base plate 138 and the dilator module 130 is coupled to the second carrier 150. In some embodiments, the dilator module 130 is movable with respect to the second carrier 150 along the insertion axis 74. The second carrier 150 includes a first mount 242 coupled to the first slide 206 and a second mount 246 coupled to the second slide 210. The first mount 242 and the second mount 246 are movable along a dilator carrier axis 250 (i.e., a transverse axis) between a first configuration (FIG. 7F) with the first slide 206 and the second slide 210 of the dilator module 130 coupled together and a second configuration (FIG. 7G) with the first slide 206 separated from the second slide 210. In other words, in the first configuration (FIG. 7F) the mounts 242, 246 are positioned together and in the second configuration (FIG. 7G) the mounts 242, 246 are spaced apart. In the illustrated embodiment, the first gear 234A and the second gear 234B are enmeshed when the mounts 242, 246 are in the first configuration. The dilator 214 is aligned with the insertion axis 74 when the first mount 242 and the second mount 246 are in the first configuration (FIG. 7F) and the dilator 214 is separated from the insertion axis 74 when the first mount 242 and the second mount 246 are in the second configuration (FIG. 7G). In some embodiments, the insertion axis 74 is positioned between the mounts 242, 246 when in the second configuration. In some embodiments, the dilator carrier axis 250 is transverse to the insertion axis 74. In some embodiments, the dilator carrier axis 250 is perpendicular to the insertion axis 74. In some embodiments, the dilator carrier axis 250 is parallel to the needle carrier axis 198. In the illustrated embodiment, the first slide 206 is movable with respect to the first mount 242 along the insertion axis 74 (see FIG. 7E) and the first slide 206 is movable with the first mount 242 along the carrier axis 250 (see FIG. 7G).

With continued reference to FIGS. 15-17, the first slide 206 includes a groove 254 through which the guidewire 122 is allowed to pass as the first slide 206 moves along the dilator carrier axis 250 to the second configuration. The guidewire 122 is positioned within the groove 254 (FIGS. 15 and 16) when the first mount 242 and the second mount 246 are in the first configuration. The guidewire 122 is spaced from the groove 254 (FIG. 17) when the first mount 242 and the second mount 246 are in the second configuration and the slides 206, 210 are spaced apart.

With reference to FIGS. 7A-7G, the deployment assembly 62 includes a guidewire drive wheel 258 that is removably coupled to the actuator 89. In the illustrated embodiment, the guidewire 122 is positioned between the drive wheel 258 and an idler wheel 262. In the illustrated embodiment, the base plate 138 is positioned between the guidewire drive wheel 258 and the insertion axis 74. In other words, the guidewire drive wheel 258 (and the actuator 89) is positioned beneath the base plate 138, and the insertion axis 74 is above the base plate 138 in the illustrated embodiment. Actuation of the guidewire drive wheel 258 by the actuator 89 moves the guidewire 122 along the insertion axis 74. In the illustrated embodiment, the guidewire 122 can be moved along the insertion axis 74 by the guidewire drive wheel 258 in both directions. As such, the guidewire actuator 89 is controlled to automatically position the guidewire 122 appropriately for the medical procedure being performed.

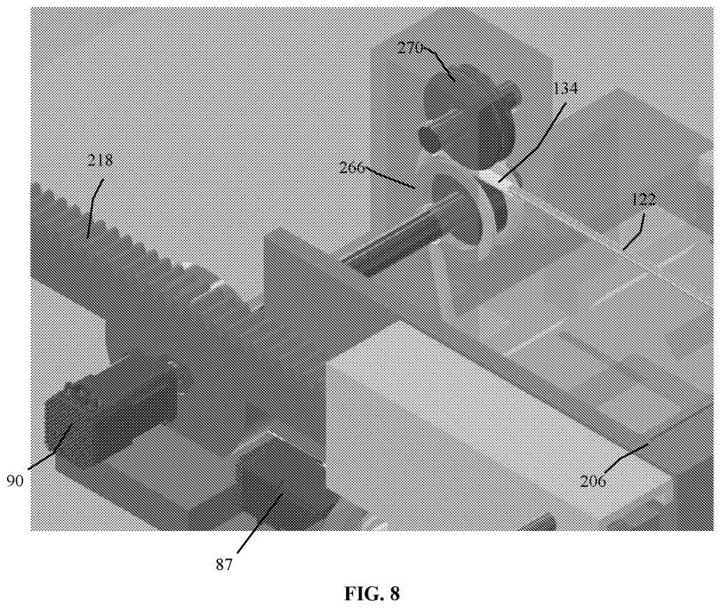

With reference to FIG. 8, the deployment assembly 62 includes a catheter drive wheel 266 that is removably coupled to the actuator 90. In the illustrated embodiment, the catheter 134 is positioned between the drive wheel 266 and an idler wheel 270. Actuation of the catheter drive wheel 266 by the actuator 90 moves the catheter 134 along the insertion axis 74. In the illustrated embodiment, the catheter 134 can be moved along the insertion axis 74 by the catheter drive wheel 266 in both directions. In the illustrated embodiment, the guidewire 122 is positioned within the catheter 134 and the guidewire 122 and the catheter 134 are independently movable. As such, the catheter actuator 90 is controlled to automatically position the catheter 134 appropriately for the medical procedure being performed.

In operation of the illustrated embodiment, the deployment assembly 62 is inserted into the cutout 66 of the deployment device 10, and the deployment device 10 is positioned with respect to a patient. At this initial starting point, the guidewire 122 is threaded inside the needle 162, the dilator 214, and the catheter 134 and the modules 126, 130 are in the starting positions shown in FIG. 7A. Next, the needle module 126 and the guidewire 122 move forward along the insertion axis 74 toward a blood vessel of a patient (FIG. 7B). Then, the needle module 126 and the guidewire 122 stop after entering the blood vessel. Next, the needle module 126 is moved backwards along the insertion axis 74 back to the starting position (FIG. 7C). At this point, the catheter 134 is still in the starting position. Next, the needle module 126 moves to an open configuration and splits into two parts, moving to the sides along the needle carrier axis 198 (FIG. 7D). The guidewire 122 and the catheter 134 remains in position as the needle module 126 separates. Next, the dilator module 130 moves forward along the insertion axis 74 and enters the blood vessel (FIG. 7E). Next, the dilator module 130 is moved backwards along the insertion axis 74 bask to the starting position (FIG. 7F). At this point, the catheter 134 is still in the starting position. Next, the dilator module 130 moves to an open configuration and splits into two parts, moving to the sides along the dilator carrier axis 250 (FIG. 7G). Next, the catheter 134 is moved forward over the guide wire 122 along the insertion axis 74 until the catheter 134 is inserted into the blood vessel. The drive wheel 266 moves the catheter 134 relative to the guidewire 122. Next, the guidewire 122 is retracted from the blood vessel as the catheter 134 remains in position. The drive wheel 138 moves the guidewire 122 relative to the catheter 134. At this point, the catheter 134 is inserted in the blood vessel and the device 10 can be removed and the deployment assembly 62 discarded. As such, the disclosure provides a method of automatically performing a medical procedure, for example, a central venous access catheter placement. As such, the deployment device 10 reduces the operating time required to obtain central venous access.

In some embodiments, a system includes the deployment device 10 and any number of deployment assemblies 62 configured for use with the deployment device 10. In some embodiments, each of the deployment assemblies is configured for a different medical procedure. In some embodiments, deployment assemblies are single use whereas the deployment device 10 is reusable.

The present disclosure provides in one aspect, a deployment device including a housing with a surface having a first aperture and a panel coupled to the housing. The panel extends from the surface at an angle and the panel includes a second aperture. The deployment device further includes a deployment assembly coupled to the housing. The deployment assembly is aligned with the first aperture and the second aperture along an insertion axis.

In some embodiments, an actuator is configured to move a portion of the deployment assembly relative to the housing. In some embodiments, the actuator is configured to move the portion along the insertion axis. In some embodiments, the actuator is configured to move the portion along a transverse axis that intersects the insertion axis. In some embodiments, the transverse axis is perpendicular to the insertion axis.

In some embodiments, the actuator is a first actuator and the portion a first portion, and wherein the device further includes a second actuator configured to move a second portion of the deployment assembly along a transverse axis that is perpendicular to the insertion axis.

In some embodiments, the actuator is a first actuator and the portion a first portion, and wherein the device further includes a second actuator configured to move a second portion of the deployment assembly relative to the housing.

In some embodiments, the actuator is an electric motor and the device further includes a power supply positioned within the housing.

In some embodiments, the housing includes a slot, wherein a portion of the deployment assembly is movable relative to the housing within the slot.

In some embodiments, the angle is within a range of 30 degree to 50 degrees. In some embodiments, the angle is 40 degrees.

In some embodiments, the deployment assembly is a peripheral venous access deployment module. In some embodiments, the deployment assembly is a central venous access deployment module. In some embodiments, the central venous access deployment module includes a needle, a dilator, a guidewire, and a catheter.

In some embodiments, the deployment device includes a processor and a memory, wherein the memory includes instructions that when executed by the processor position the catheter within a vein of a patient.

In some embodiments, the deployment assembly is a first deployment assembly and the first deployment assembly is removable from the housing and replaceable with a second deployment assembly. In some embodiments, the actuator is coupled to the deployment assembly by a detachable interface. In some embodiments, the detachable interface includes a groove formed in the actuator and a drive shaft in the deployment assembly at least partially positioned within the groove.

In some embodiments, the deployment device further includes an ultrasound module coupled to the panel. In some embodiments, the deployment device further includes a handle coupled to the housing.

The present disclosure provides in one aspect, an assembly including a base module with a base plate and a carrier movable with respect to the base plate. The assembly further includes a guidewire extending along an insertion axis and movable with respect to the base plate along the insertion axis. The assembly further includes a needle module coupled to the carrier, and a catheter movable with respect to the base plate along the insertion axis.

In some embodiments, the needle module is movable with the carrier, and movable with respect to the carrier along the insertion axis. In some embodiments, the needle module includes a slide, a needle coupled to the slide, and a rack coupled to the slide.

In some embodiments, the assembly further includes an actuator and a transmission positioned between the actuator and the rack, and wherein the actuator is activated to move the needle along the insertion axis.

In some embodiments, the needle is movable between a closed configuration and an open configuration. In some embodiments, the assembly further includes an actuator and a transmission positioned between the actuator and the needle, wherein the actuator is activated to move the needle between the closed configuration and the open configuration. In some embodiments, the needle includes an outer cylindrical member with a first slot and an inner cylindrical member with a second slot, and wherein the first slot and the second slot are aligned when the needle is in the open configuration.

In some embodiments, the slide includes a first slide and a second slide separable from the first slide, and wherein the needle is coupled to the first slide and the transmission is coupled to the second slide. In some embodiments, the carrier includes a first carrier portion coupled to the first slide and a second carrier portion coupled to the second slide, wherein the first carrier portion and the second carrier portion are movable along a carrier axis between a first configuration with the first slide and the second slide coupled together and a second configuration with the first slide separated from the second slide. In some embodiments, the carrier axis is transverse to the insertion axis.

In some embodiments, the needle includes an outer cylindrical member and an inner cylindrical member positioned within the outer cylindrical member. The outer cylindrical member and the inner cylindrical member are aligned with the insertion axis when the first carrier portion and the second carrier portion are in the first configuration, and the outer cylindrical member and the inner cylindrical member are separated from the insertion axis when the first carrier portion and the second carrier portion are in the second configuration.

In some embodiments, the outer cylindrical member includes a first slot and the inner cylindrical member includes a second slot, and wherein the first slot and the second slot are aligned when the needle is in the open configuration. In some embodiments, the first slide includes a groove and wherein the guidewire is positioned within the groove when the first carrier portion and the second carrier portion are in the first configuration.

In some embodiments, the carrier is a needle carrier, and the assembly further includes a dilator carrier movable with respect to the base plate and a dilator module coupled to the dilator carrier. In some embodiments, the dilator carrier defines a dilator carrier axis and the dilator module is movable along the insertion axis and movable along the dilator carrier axis. In some embodiments, the dilator module includes a dilator with an outer cylindrical member and an inner cylindrical member positioned within the outer cylindrical member. The dilator is movable between a closed configuration and an open configuration.

In some embodiments, the assembly further includes a guidewire drive wheel.

Actuation of the guidewire drive wheel moves the guidewire along the insertion axis. In some embodiments, the base plate is positioned between the guidewire drive wheel and the insertion axis. In some embodiments, the assembly further includes a catheter drive wheel. Actuation of the catheter drive wheel moves the catheter along the insertion axis. In some embodiments, the guidewire is positioned within the catheter.

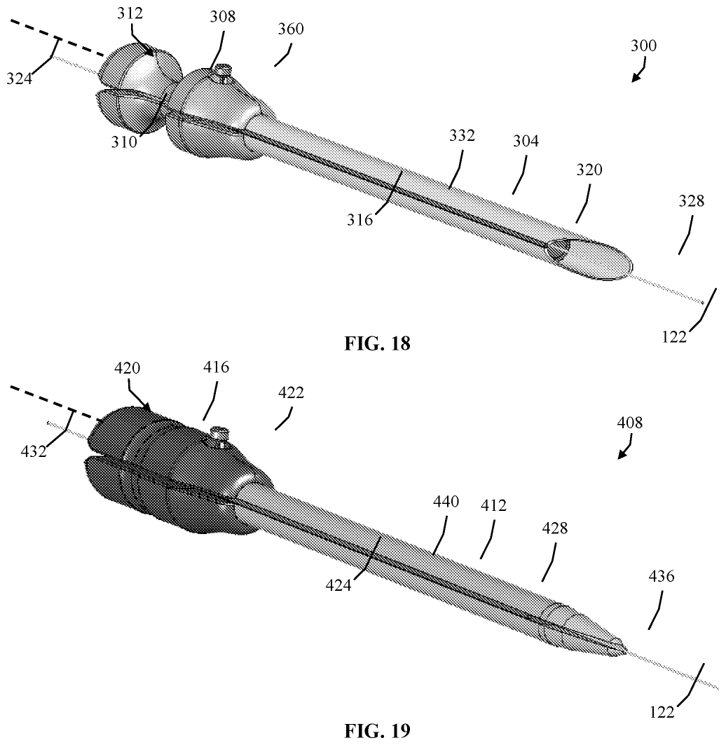

With reference to FIG. 18, a needle 300 is illustrated as a stand-alone device. The needle 300 is similar to the needle 162 described in relation to the needle module 126 and details herein apply to both the needle 300 and the needle 162. The needle 300 includes a cylindrical member 304, a handle 308 coupled to a proximal end 312 of the cylindrical member 304, and a blocking member 316 movable with respect to the cylindrical member 304 between a closed configuration and an open configuration.

The cylindrical member 304 includes a cylindrical wall 320 extending along a longitudinal axis 324. In the illustrated embodiment, the cylindrical member 304 includes a beveled distal end 328. A first slot 332 is formed in the cylindrical wall 320 and the first slot 332 extends along the longitudinal axis 324. In the closed configuration, the first slot 332 is blocked by the blocking member 316 and in the open configuration the first slot 332 is open to the longitudinal axis 324. In other words, the needle 300 can be positioned around a guidewire (e.g., the guidewire 122) in the open configuration and the needle 300 is secured around the guidewire in the closed configuration. Similar to the needle 162 of deployment assembly 62, the needle 300 is advantageously laterally positioned around the guidewire as opposed to threading the needle 300 along the length of the guidewire.

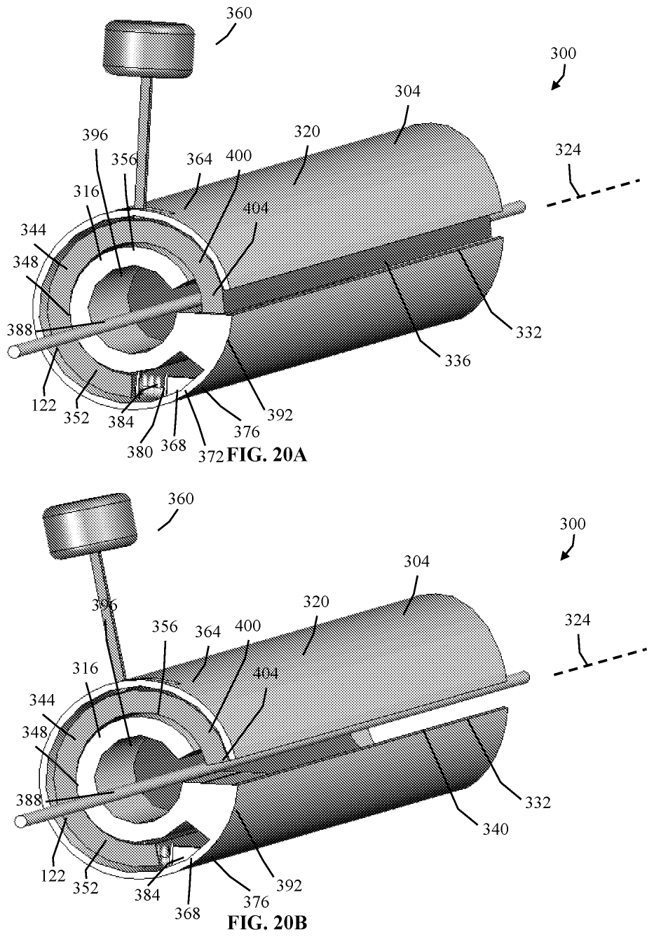

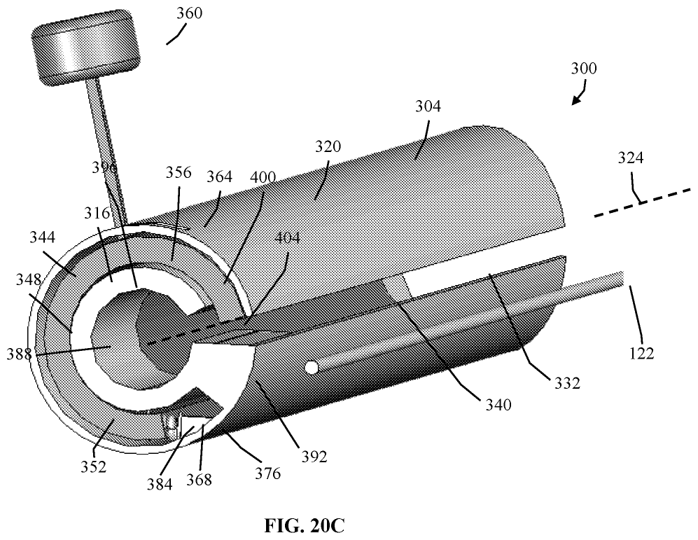

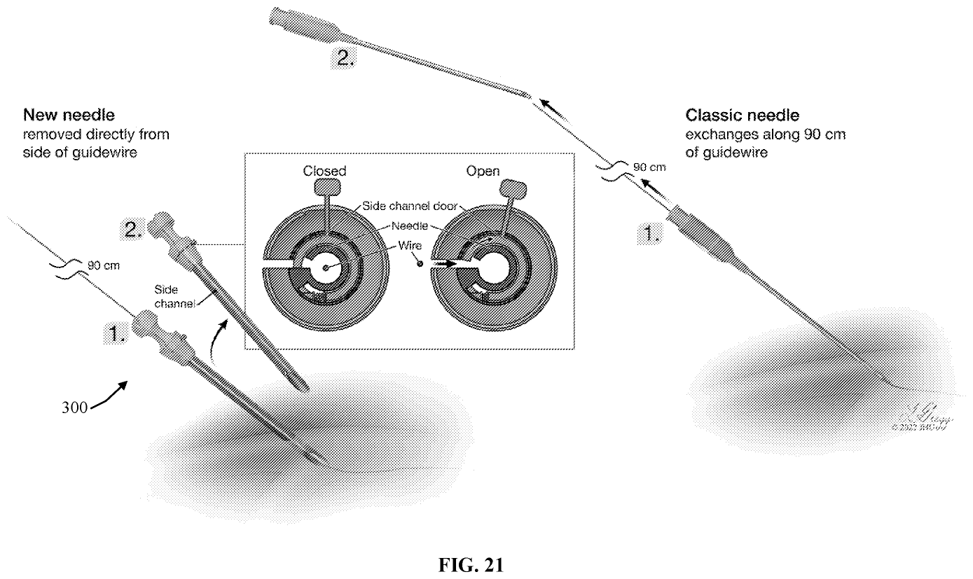

With reference to FIGS. 20A-20C, in the illustrated embodiment, the cylindrical member 304 is a first cylindrical member and the cylindrical wall 320 is a first cylindrical wall, and the blocking member 316 is a second cylindrical member with a second cylindrical wall 336 extending along the longitudinal axis 324. In the illustrated embodiment, the two cylindrical members 304, 316 of the needle 300 are concentric. In some embodiments, the blocking member 316 is any suitable shape to at least partially block the slot 332 in the first cylindrical member 304.

In the illustrated embodiment, the second cylindrical wall 336 is positioned within the first cylindrical wall 320. The second cylindrical member 316 includes a second slot 340. In the illustrated embodiment, the needle 300 includes an outer cylindrical member 304 with a first slot 332 and an inner cylindrical member 316 with a second slot 340. The first cylindrical wall 320 includes a first outer surface 344 and a first inner surface 348. The first slot 332 extends between the first outer surface 344 and the first inner surface 348. The second cylindrical wall 336 includes a second outer surface 352 and a second inner surface 356. In the illustrated embodiment, the second slot 340 extends between the second outer surface 352 and the second inner surface 356. In the illustrated embodiment, the first inner surface 348 is positioned radially between the first outer surface 344 and the second outer surface 352.

The first slot 332 and the second slot 340 are aligned in the open configuration of the needle 300 (FIGS. 20B, 20C) and the first slot 332 and the second slot 340 are misaligned in the closed configuration of the needle 300 (FIG. 20A). In other words, a portion of the second slot 340 overlaps the first slot 332 in the open configuration, and a portion of the cylindrical wall 336 overlaps the first slot 332 in the closed configuration. The handle 308 includes a handle slot 310 aligned with the first slot 332 of the first cylindrical member 304.

With continued reference to FIG. 18, the needle 300 includes an actuator 360. In the illustrated embodiment, the actuator 360 is integrally formed with the second cylindrical member 316 and the actuator 360 radially extends from the second cylindrical wall 336. The actuator 360 extends through an aperture 364 formed in the first cylindrical wall 320. In the illustrated embodiment, the actuator 360 extends through the handle 308 and is graspable and actuated by a user.

With reference to FIG. 20A, the needle 300 includes a biasing member 368 positioned between the first cylindrical member 304 and the second cylindrical member 316. The biasing member 368 biases the second cylindrical member 316 toward the closed configuration. In other words, the biasing member 368 is positioned between the first cylindrical member 304 and the blocking member 316 and biases the blocking member 316 toward the closed configuration. In the illustrated embodiment, the biasing member 368 is a coil spring. In other embodiments, the biasing member 368 is a torsion spring or other suitable biasing element. A first end 372 of the biasing member 368 abuts a stop 376 formed on the first cylindrical member 304, and a second end 380 of the biasing member 368 abuts a radial wall 384 of the second cylindrical member 316. In the illustrated embodiment, the radial wall 384 at least partially defines the second slot 340.

With reference to FIGS. 20A-20C, the first cylindrical member 304 includes a support hub 388. In the illustrated embodiment, the support hub 388 is slidably coupled to the second cylindrical member 316. The support hub 388 includes a radial portion 392 extending radially inward from the first cylindrical wall 320 and a bearing portion 396. The radial portion 392 extends through the second cylindrical wall 336. In the illustrated embodiment, the bearing portion 396 includes a cylindrical wall 400 with a slot 404. The bearing portion 396 rotatably supports the second cylindrical member 316 to permit the second cylindrical member 316 to rotate about the axis 324 with respect to the first cylindrical member 304.

With reference to FIG. 19, a dilator 408 is illustrated as a stand-alone device. The dilator 408 is similar to the needle 300 of FIG. 18 and description relating to the structure and operation of the needle 300 apply similarly to the dilator 408. In addition, the dilator 408 is similar to the dilator 214 described in relation to the dilator module 130 and details herein apply to both the dilator 408 and the dilator 214.

The dilator 408 includes a cylindrical member 412, a handle 416 coupled to a proximal end 420 of the cylindrical member 412, and a blocking member 424 movable with respect to the cylindrical member 412 between a closed configuration and an open configuration. In the illustrated embodiment, an actuator 422 moves the blocking member 424. The cylindrical member 412 includes a cylindrical wall 428 extending along a longitudinal axis 432. In the illustrated embodiment, the cylindrical member 412 includes a conical distal end 436. A first slot 440 is formed in the cylindrical wall 428 and the first slot 440 extends along the longitudinal axis 432. In the closed configuration, the first slot 440 is blocked by the blocking member 424 and in the open configuration the first slot 440 is open to the longitudinal axis 432. In other words, the dilator 408 can be positioned around a guidewire (e.g., the guidewire 122) in the open configuration and the dilator 408 is secured around the guidewire in the closed configuration. Similar to the dilator 214, the dilator 408 is advantageously laterally positioned around the guidewire as opposed to threading the dilator 408 along the length of the guidewire.

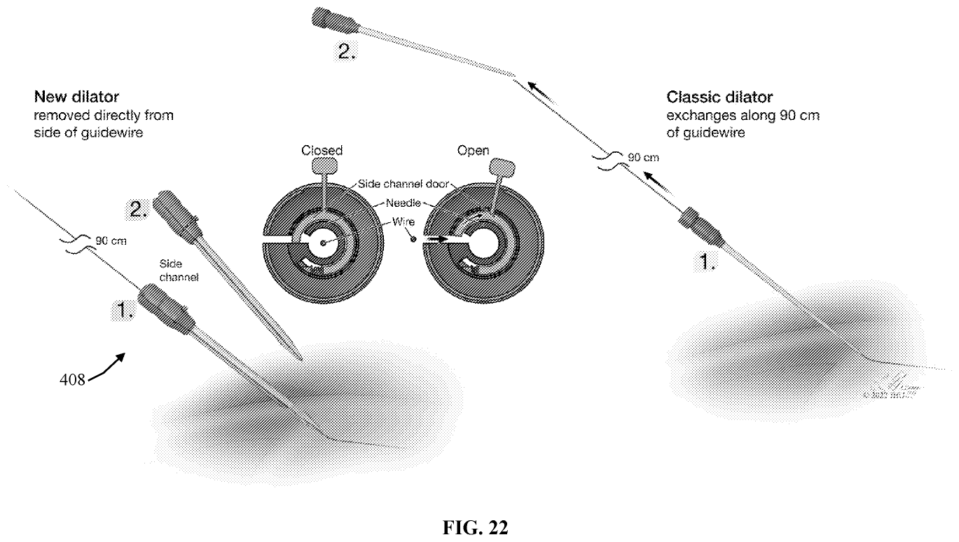

In operation, with reference to FIGS. 20A-20C, in the illustrated embodiment, the needle 300 is biased to the closed configuration (FIG. 20A) by the biasing member 368. In the closed configuration, the guidewire 122 is secured within the needle 300. The actuator 360 is actuated by a user to move the needle 300 to the open configuration (FIG. 20B). Specifically, the actuator 360 rotates the inner cylindrical member 316 about the longitudinal axis 324 with respect to the outer cylindrical member 304 against the bias of biasing member 368. In the open configuration, the needle 300 is configured to radially receive the guidewire 122 through the slots 332, 340. Likewise, in the open configuration, the needle 300 is configured to be laterally separate from the guidewire 122 though the slots 332, 340. With reference to FIG. 21, advantageous operation of the needle of FIG. 18 is shown compared to operation of a conventional needle. With reference to FIG. 22, advantageous operation of the dilator of FIG. 19 is shown compared to operation of a conventional dilator.

The present disclosure provides in one aspect, a device including a cylindrical member with a cylindrical wall extending along a longitudinal axis. A slot is formed in the cylindrical wall and the slot extends along the longitudinal axis. The device further includes a blocking member movable with respect to the cylindrical member between a closed configuration in which the slot is blocked by the blocking member and an open configuration in which the slot is open to the longitudinal axis.

In some embodiments, the cylindrical member is a first cylindrical member and the cylindrical wall is a first cylindrical wall. The blocking member is a second cylindrical member with a second cylindrical wall extending along the longitudinal axis. In some embodiments, the second cylindrical wall is positioned within the first cylindrical wall.

In some embodiments, the slot is a first slot and the second cylindrical member includes a second slot. The first slot and the second slot are aligned in the open configuration, and the first slot and the second slot are misaligned in the closed configuration.

In some embodiments, the first cylindrical wall includes a first outer surface and a first inner surface, and the first slot extends between the first outer surface and the first inner surface. The second cylindrical wall includes a second outer surface and a second inner surface, and the second slot extends between the second outer surface and the second inner surface. In some embodiments, the first inner surface is positioned between the first outer surface and the second outer surface. In some embodiments, the second cylindrical member includes an actuator radially extending from the second cylindrical wall. In some embodiments, the actuator extends through an aperture formed in the first cylindrical wall.

In some embodiments, the device further includes a handle coupled to an end of the first cylindrical member, and the actuator extends through the handle. In some embodiments, the handle includes a handle slot aligned with the first slot.

In some embodiments, the device further includes a biasing member positioned between the first cylindrical member and the second cylindrical member. The biasing member biases the second cylindrical member toward the closed configuration. In some embodiments, a first end of the biasing member abuts a stop formed on the first cylindrical member, and a second end of the biasing member abuts a radial wall of the second cylindrical member.

In some embodiments, the device further includes a biasing member positioned between the cylindrical member and the blocking member. The biasing member biases the blocking member toward the closed configuration. In some embodiments, the first cylindrical member includes a support hub coupled to the second cylindrical member.

In some embodiments, the support hub includes a radial portion extending radially inward from the first cylindrical wall and a bearing portion. In some embodiments, the radial portion extends through the second cylindrical wall.

In some embodiments, the device is a needle. In some embodiments, the cylindrical member includes a beveled distal end. In some embodiments, the device is a dilator. In some embodiments, the cylindrical member includes a conical distal end. In some embodiments, the device in the open configuration is configured to radially receive a guidewire through the slot.

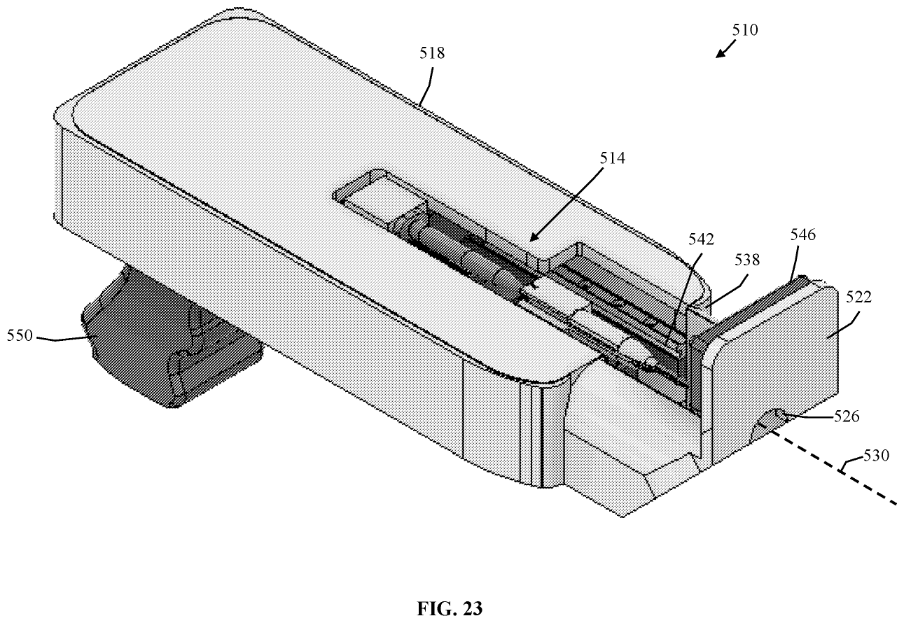

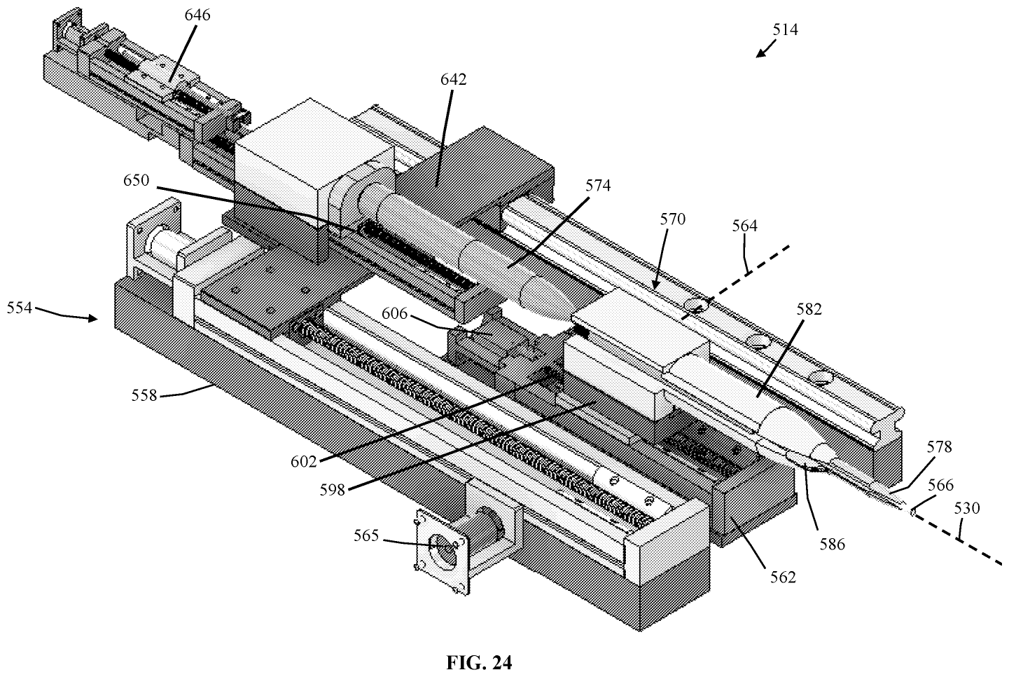

With reference to FIG. 23, a deployment device 510 including a deployment assembly 514 is illustrated. The deployment device 510 includes a housing 518 and a panel 522 coupled to the housing 518. The panel 522 includes an aperture 526 that is aligned with an insertion axis 530. The deployment assembly 514 is removably coupled to the housing 518. The deployment assembly 514 is aligned with the aperture 526 along the insertion axis 530.

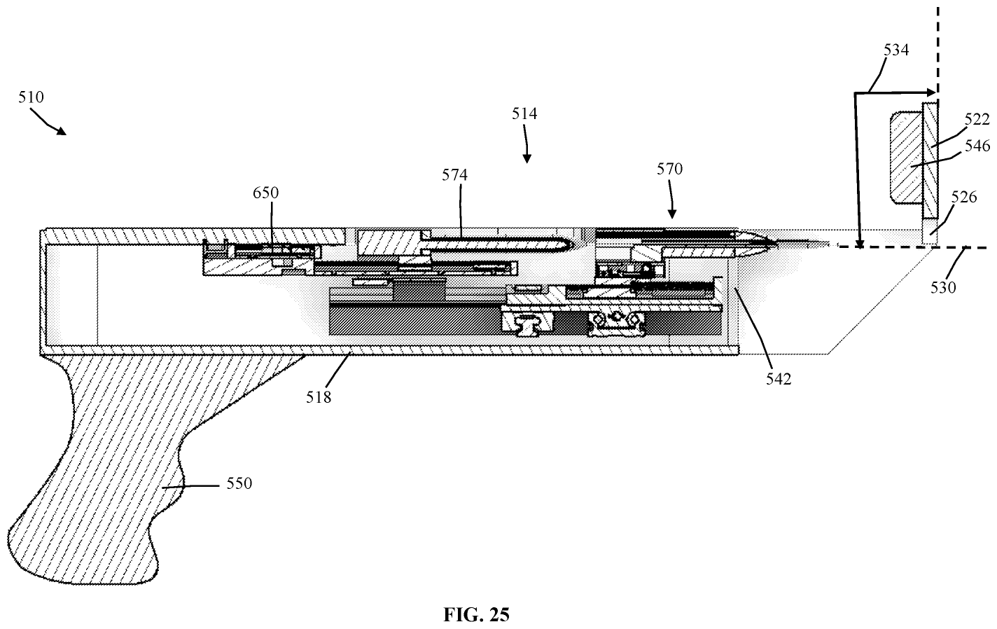

With reference to FIG. 25, the panel 522 extends from the housing 518 at an angle 534. In some embodiments, the angle 534 is within a range of approximately 30 degrees to approximately 90 degrees. In the illustrated embodiment, the angle 534 is approximately 90 degrees.

With reference to FIGS. 24 and 25, the deployment device 510 includes an actuator (e.g., actuator 606) configured to move a portion of the deployment assembly 514 relative to the housing 518. In some embodiments, the actuator is configured to move the portion along the insertion axis 530. In other embodiments, the actuator is configured to move the portion along a transverse axis that intersects the insertion axis 530. In some embodiments, the transverse axis is perpendicular to the insertion axis 530. In some embodiments, the actuator is one of a plurality of actuators. In some embodiments, the actuator is a first actuator and the portion is a first portion, and the deployment device 510 further includes a second actuator (e.g., actuator 565) configured to move a second portion of the deployment assembly 514 along a transverse axis that is perpendicular to the insertion axis 530. In some embodiments, a second actuator is configured to move a second portion of the deployment assembly relative to the housing 518. In some embodiments, the actuator is an electric motor and the deployment device 510 further includes a power supply positioned within the housing. In some embodiments, the actuator is coupled to the deployment assembly by a detachable interface (e.g., a tongue and groove configuration).

With reference to FIG. 23, the housing 518 includes a surface 538 (e.g., a front surface) with an opening 542. The panel 522 extends from the surface 538 and the opening 542 is aligned with the aperture 526. In other words, the insertion axis 530 extends through the aperture 526 and the opening 542. In the illustrated embodiment, an ultrasound module 546 is coupled to the panel 522. In the illustrated embodiment, the device 510 further includes a handle 550 coupled to the housing 518.

In some embodiments, the deployment assembly 514 is a peripheral venous access deployment module. In other embodiments, the deployment assembly 514 is a central venous access deployment module. The deployment assembly 514 is a first deployment assembly and is removable from the housing 518 and replaceable with a second deployment assembly. In other words, the deployment assembly is replaceable. In some embodiments, at least some portions of the deployment assembly are single use disposable components.

With reference to FIG. 24, the deployment assembly 514 includes a base 554 with a frame 558 and a carrier 562 movable with respect to the frame 558. The deployment assembly 514 further includes a guidewire 566 extending along the insertion axis 530 and movable with respect to the frame 558 along the insertion axis 530. The deployment device 510 includes a needle module 570 coupled to the carrier 562, and a catheter 574 movable with respect to the frame 558 along the insertion axis 530. The needle module 570 is movable with the carrier 562, and movable with respect to the carrier 562 along the insertion axis 530. In the illustrated embodiment, the needle module 570 is mounted on the carrier 562 and portions of the needle module 570 are movable independent of the carrier 562.

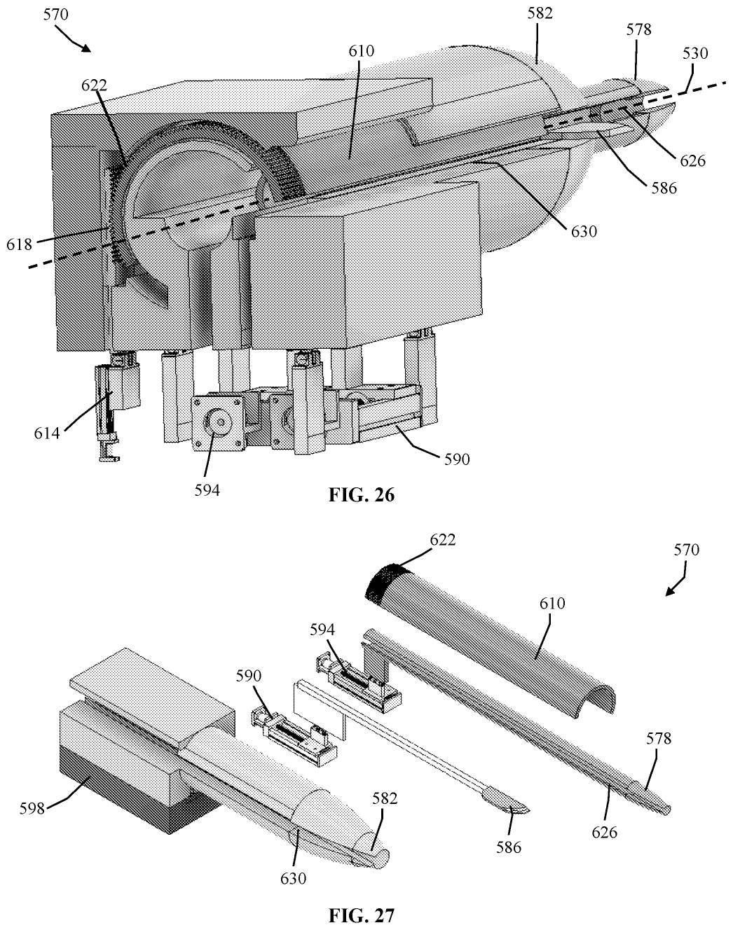

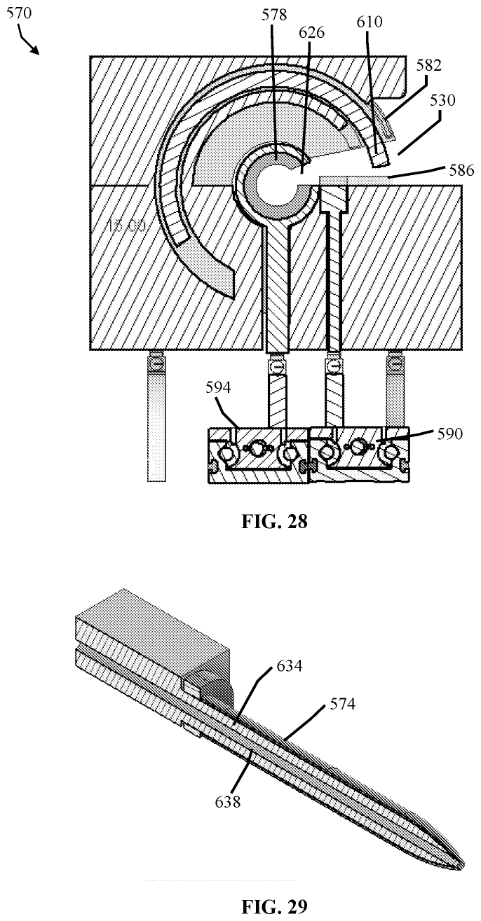

With reference to FIG. 26, the needle module 570 includes a needle 578, a dilator 582, and a knife 586. In the illustrated embodiment, the needle 578 and the knife 586 are at least partially positioned within the dilator 582. With reference to FIGS. 27 and 28, the needle module 570 further includes a knife drive assembly 590 coupled to the knife 586, and a needle drive assembly 594 coupled to the needle 578. In the illustrated embodiment, the knife 586 and the needle 578 are independently movable along the insertion axis 530 with respect to the dilator 582. In some embodiment, the knife 586 and the needle 578 are independently movable with respect to the guidewire 566. In the illustrated embodiment, the knife 586 is a retractable knife. The needle 578 is independently movable with respect to the dilator 582 and the knife 586 by activation of the needle drive assembly 594. Likewise, the knife 586 is independently movable with respect to the needle 578 and the dilator 582 by activation of the knife drive assembly 590.

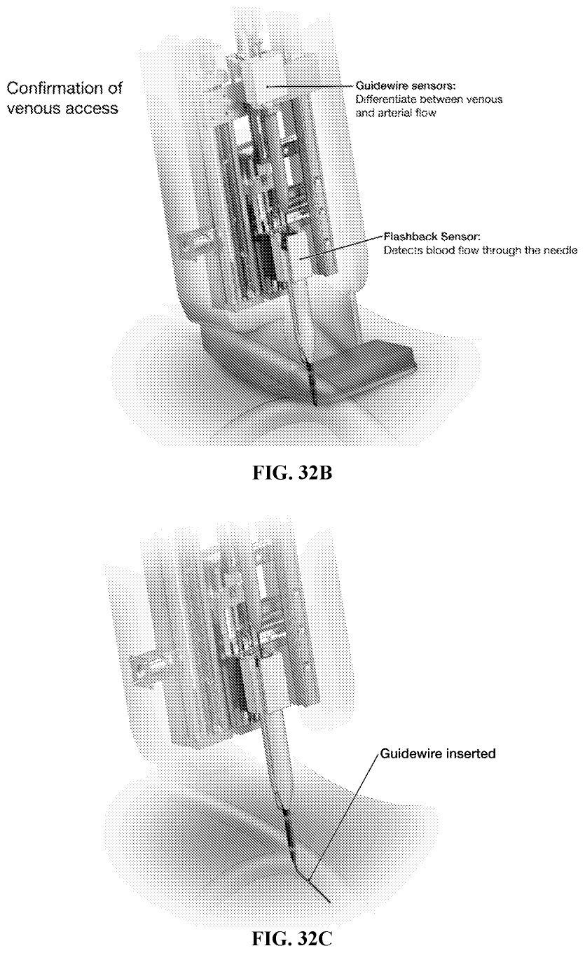

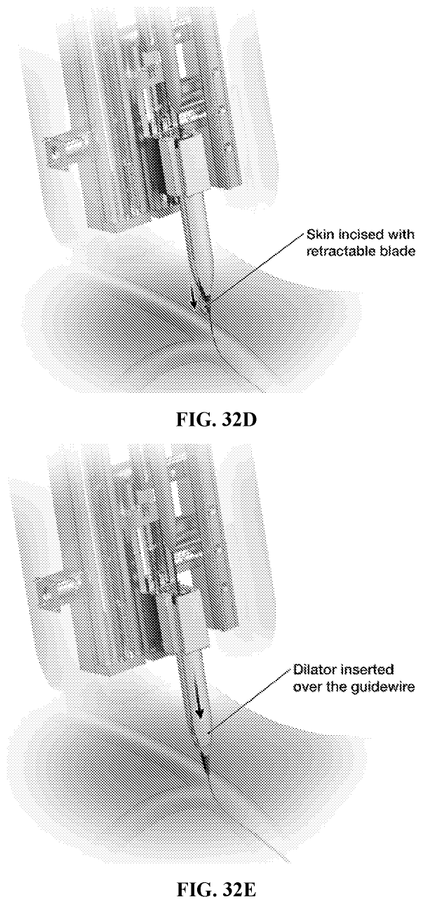

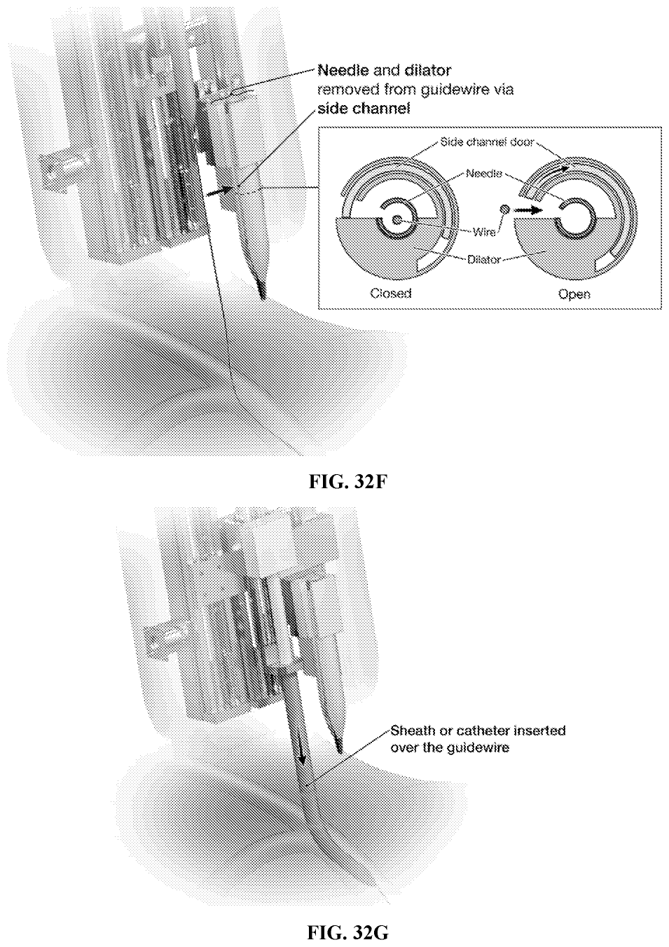

With reference to FIG. 24, the needle module 570 includes a slide 598 and the needle 578, the dilator 582, and the knife 586 are coupled to the slide 598. A transmission 602 positioned between an actuator 606 and the slide 598 is configured to move the slide 598 along the insertion axis 530. In the illustrated embodiment, the actuator 606 is activated to move the slide 598, the needle 578, the dilator 582, and the knife 586 along the insertion axis 530. In other words, the actuator 606 is activated to move the needle module 570 relative to the carrier 562 along the insertion axis 530. As detailed herein, the carriage 562, and the needle module 570 coupled to the carriage 562, moves relative to the frame 558 in a direction 564 transverse to the insertion axis 530. Activation of an actuator 565 moves the carriage 562 in the transverse direction 564. In the illustrated embodiment, the carriage 562 and the needle module 570 move perpendicular to the insertion axis 530 after operation with the needle module 570 is complete (FIG. 32F).

With reference to FIGS. 26-28, the needle module 570 further includes a cover 610 movable between a closed configuration and an open configuration. With reference to FIG. 26, the needle module 570 further includes a cover actuator 614 coupled to the cover 610. The cover actuator 614 is activated to move the cover 610 between the closed configuration and the open configuration. In the illustrated embodiment, the cover actuator 614 includes a rack portion 618 engaged with teeth 622 formed on the cover 610 to cause the cover 610 to rotate about the insertion axis 530. The needle 578 includes a needle slot 626 and the dilator 582 includes a dilator slot 630. The dilator slot 630 is aligned with the needle slot 626. In the open configuration, the cover 610 is spaced from the needle slot 626 and the dilator slot 630. In other words, the cover 610 does not block the slots 626, 630 in the open configuration. In the closed configuration, the cover 610 is positioned to block slots 626, 630, which prevents the guidewire 566 from moving through the slots 626, 630.

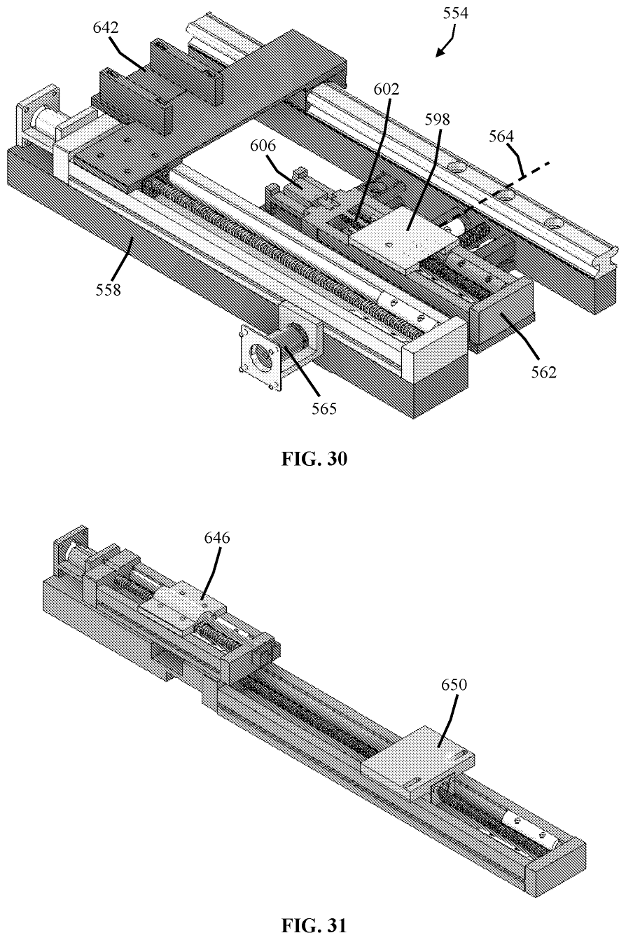

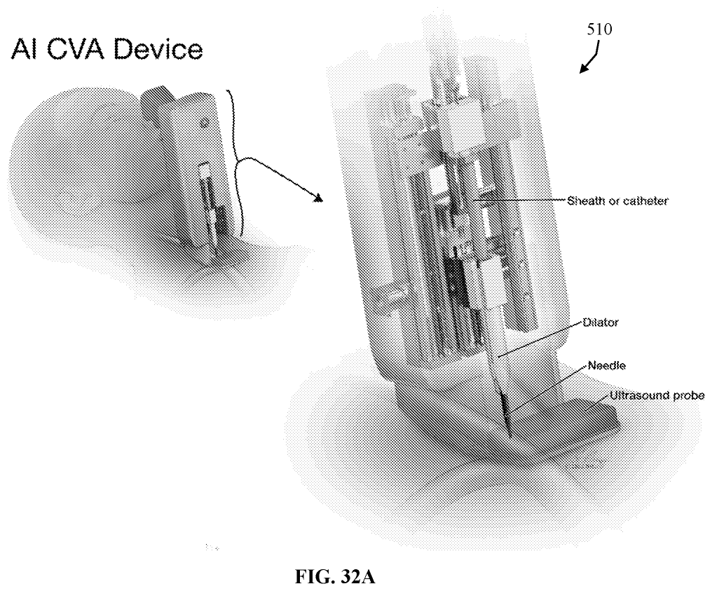

With reference to FIG. 29, the catheter 574 is position on a support 634 with a passageway 638 that receives the guidewire 566. In the illustrated embodiment, the guidewire 566 is positioned within the catheter 574. With reference to FIG. 30, the base 554 further includes a second carrier 642 movable with respect to the frame 558 along the insertion axis 530. With reference to FIG. 31, a guidewire drive assembly 646 and a catheter drive assembly 650 are mounted on to the second carrier 642. The guidewire drive assembly 646 is configured to move the guidewire 566 along the insertion axis 530, and the catheter drive assembly 650 is configured to move the catheter 574 along the insertion axis 530. Actuation of the guidewire drive assembly 646 moves the guidewire 566 along the insertion axis 530 with respect to the frame 558. Likewise, actuation of the catheter drive assembly 650 moves the catheter 574 and the catheter support 634 along the insertion axis 530 with respect to the frame 558.

With reference to FIGS. 32A-32G, operation of the deployment device 510 is illustrated. FIG. 32A illustrates positioning the deployment device 510 with respect to a patient. FIG. 32B illustrates the needle 578 being inserted into the vein. FIG. 32C illustrates the guidewire 566 being inserted into the vein. FIG. 32D illustrates the knife 586 cutting the skin. FIG. 32E illustrates the dilator 582 being inserted into through the cut skin. FIG. 32F illustrates the needle module 570 moving from the closed configuration to the open configuration. FIG. 32F also illustrates the needle module 570 moving on the carrier 562 to the side, away from the insertion axis 530, once the needle module 570 is in the open configuration. FIG. 32G illustrates the catheter 574 being inserted into the vein over the guidewire 566. As detailed herein, the deployment device includes a processor and a memory. The memory includes instructions that when executed by the processor, position the catheter within a vein of a patient.

One skilled in the art will readily appreciate that the present disclosure is well adapted to carry out the objects and obtain the ends and advantages mentioned, as well as those inherent herein. The present disclosure described herein are exemplary embodiments and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Various features and advantages are set forth in the following claims.

What is claimed is:

1. A deployment device comprising:
a housing;
a panel coupled to the housing, wherein the panel extends from the housing at an angle and the panel includes an aperture; and
a deployment assembly coupled to the housing;
wherein deployment assembly is aligned with the aperture along an insertion axis.

2. The device of claim 1, further comprising an actuator configured to move a portion of the deployment assembly relative to the housing.

3. The device of claim 2, wherein the actuator is configured to move the portion along the insertion axis.

4. The device of claim 2, wherein the actuator is configured to move the portion along a transverse axis that intersects the insertion axis.

5. The device of claim 4, wherein the transverse axis is perpendicular to the insertion axis.

6. The device of claim 3, wherein the actuator is a first actuator and the portion is a first portion, and wherein the device further includes a second actuator configured to move a second portion of the deployment assembly along a transverse axis that is perpendicular to the insertion axis.

7. The device of claim 2, wherein the actuator is a first actuator and the portion is a first portion, and wherein the device further includes a second actuator configured to move a second portion of the deployment assembly relative to the housing.

8. The device of claim 2, wherein the actuator is an electric motor and the device further includes a power supply positioned within the housing.

9. The device of claim 1, wherein the housing includes a surface with an opening, and wherein the panel extends from the surface and the opening is aligned with the aperture.

10. The device of claim 1, wherein the angle is within a range of 30 degree to 90 degrees.

11. The device of claim 10, wherein the angle is 90 degrees.

12. The device of claim 1, wherein the deployment assembly is a peripheral venous access deployment module.

13. The device of claim 1, wherein the deployment assembly is a central venous access deployment module.

14. The device of claim 13, wherein the central venous access deployment module includes a needle, a dilator, a guidewire, and a catheter.

15. The device of claim 13, wherein the central venous access deployment module includes a retractable knife.

16. The device of claim 13, further comprising a processor and a memory, wherein the memory includes instructions that when executed by the processor position the catheter within a vein of a patient.

17. The device of claim 1, wherein the deployment assembly is a first deployment assembly and the first deployment assembly is removable from the housing and replaceable with a second deployment assembly.

18. The device of claim 2, wherein the actuator is coupled to the deployment assembly by a detachable interface.

19. The device of claim 1, further comprising an ultrasound module coupled to the panel.

20. The device of claim 1, further comprising a handle coupled to the housing.

21. An assembly comprising:
a base including a frame and a carrier movable with respect to the frame;
a guidewire extending along an insertion axis and movable with respect to the frame along the insertion axis;
a needle module coupled to the carrier; and
a catheter movable with respect to the frame along the insertion axis.

22. The assembly of claim 21, wherein the needle module is movable with the carrier, and movable with respect to the carrier along the insertion axis.

23. The assembly of claim 21, wherein the needle module includes a slide, a needle coupled to the slide, an actuator, and a transmission positioned between the actuator and the slide, and wherein the actuator is activated to move the needle along the insertion axis.

24. The assembly of claim 23, wherein the actuator is activated to move the needle relative to the carrier along the insertion axis.

25. The assembly of claim 23, wherein the needle module further includes a cover movable between a closed configuration and an open configuration.

26. The assembly of claim 25, further comprising a cover actuator coupled to the cover, wherein the cover actuator is activated to move the cover between the closed configuration and the open configuration.

27. The assembly of claim 25, wherein the needle includes a slot, and wherein the cover is spaced from the slot when the needle is in the open configuration.

28. The assembly of claim 21, wherein the carriage moves relative to the frame in a direction transverse to the insertion axis.

29. The assembly of claim 21, wherein the needle module includes a knife movable along the insertion axis.

30. The assembly of claim 29, wherein the knife is movable with respect to the guidewire.

31. The assembly of claim 21, wherein the needle module further includes a dilator.

32. The assembly of claim 31, wherein the dilator is movable with respect to the guidewire along the insertion axis.

33. The assembly of claim 21, wherein the needle module includes a needle, a dilator, and a knife.

34. The assembly of claim 33, wherein the needle is independently movable with respect to the dilator and the knife, and wherein the knife is independently movable with respect to the needle and the dilator.

35. The assembly of claim 33, wherein the needle is at least partially positioned within the dilator, and wherein the knife is at least partially positioned within the dilator.

36. The assembly of claim 33, wherein the dilator includes a dilator slot aligned with a slot formed in the needle.

37. The assembly of claim 21, further including a guidewire drive, wherein actuation of the guidewire drive moves the guidewire along the insertion axis.

38. The assembly of claim 37, further including a catheter drive, wherein actuation of the catheter drive moves the catheter along the insertion axis.

39. The assembly of claim 38, further including a second carrier movable with respect to the frame along the insertion axis, wherein the guidewire drive and the catheter drive are coupled to the second carrier.

40. The assembly of claim 21, wherein the guidewire is positioned within the catheter.

41. A device comprising:
a cylindrical member with a cylindrical wall extending along a longitudinal axis, wherein a slot is formed in the cylindrical wall and the slot extends along the longitudinal axis; and
a blocking member movable with respect to the cylindrical member between a closed configuration in which the slot is blocked by the blocking member and an open configuration in which the slot is open to the longitudinal axis.

42. The device of claim 41, wherein the cylindrical member is a first cylindrical member and the cylindrical wall is a first cylindrical wall, and wherein the blocking member is a second cylindrical member with a second cylindrical wall extending along the longitudinal axis.

43. The device of claim 42, wherein the second cylindrical wall is positioned within the first cylindrical wall.

44. The device of claim 42, wherein the slot is a first slot and the second cylindrical member includes a second slot; wherein the first slot and the second slot are aligned in the open configuration, and wherein the first slot and the second slot are misaligned in the closed configuration.

45. The device of claim 44, wherein the first cylindrical wall includes a first outer surface and a first inner surface, wherein the first slot extends between the first outer surface and the first inner surface, and wherein the second cylindrical wall includes a second outer surface and a second inner surface, wherein the second slot extends between the second outer surface and the second inner surface.

46. The device of claim 45, wherein the first inner surface is positioned between the first outer surface and the second outer surface.

47. The device of claim 46, wherein the second cylindrical member includes an actuator radially extending from the second cylindrical wall.

48. The device of claim 47, wherein the actuator extends through an aperture formed in the first cylindrical wall.

49. The device of claim 47, further comprising a handle coupled to an end of the first cylindrical member, wherein the actuator extends through the handle.

50. The device of claim 49, wherein the handle includes a handle slot aligned with the first slot.

51. The device of claim 42, further including a biasing member positioned between the first cylindrical member and the second cylindrical member, wherein the biasing member biases the second cylindrical member toward the closed configuration.

52. The device of claim 51, wherein a first end of the biasing member abuts a stop formed on the first cylindrical member, and a second end of the biasing member abuts a radial wall of the second cylindrical member.

53. The device of claim 41, further including a biasing member positioned between the cylindrical member and the blocking member, wherein the biasing member biases the blocking member toward the closed configuration.

54. The device of claim 42, wherein the first cylindrical member includes a support hub coupled to the second cylindrical member.

55. The device of claim 54, wherein the support hub includes a radial portion extending radially inward from the first cylindrical wall and a bearing portion.

56. The device of claim 55, wherein the radial portion extends through the second cylindrical wall.

57. The device of claim 41, wherein the device is a needle.

58. The device of claim 57, wherein the cylindrical member includes a beveled distal end.

59. The device of claim 41, wherein the device is a dilator.

60. The device of claim 59, wherein the cylindrical member includes a conical distal end.

61. The device of claim 41, wherein the device in the open configuration is configured to radially receive a guidewire through the slot.
